(19) United States
(12) Patent Application Publication
(10) Pub. No.: US 2011/0064750 A1
(43) Pub. Date: Mar. 17, 2011

(54) METHOD AND KIT FOR TREATING NICOTINE ADDICTION

(75) Inventors: Raafat Fahim, Boca Raton, FL (US); Ali Fattom, Rockville, MD (US); Paul Kessler, Hagerstown, MD (US); Matthew Kalnik, Bethesda, MD (US)

(73) Assignee: Nabi Biopharmaceuticals

(21) Appl. No.: 12/846,514

(22) Filed: Jul. 29, 2010

Related U.S. Application Data
(60) Provisional application No. 61/230,301, filed on Jul. 31, 2009.

(51) Int. Cl. A61K 39/395; A61P 25/34

(52) U.S. Cl. 424/178.1; 424/130.1

(57) ABSTRACT

Described are methods and kits related to treating nicotine addiction and increasing the likelihood of nicotine abstinence. Methods and kits for reestablishing nicotine abstinence after a relapse to nicotine use are also described.

### RELATED APPLICATIONS

This application claims the benefit of priority under 35 U.S.C. §119(e) to U.S. provisional application 61/230,301, filed Jul. 31, 2009, the entire contents of which are incorporated herein by reference.

### FIELD OF THE INVENTION

The present invention relates to the field of smoking cessation and provides methods and kits for smoking cessation.

### BACKGROUND

Smoking and tobacco use is a global healthcare problem. The World Health Organization estimates that there are 1.3 billion smokers worldwide today and nearly five million tobacco-related deaths each year. If current smoking patterns continue, smoking will cause some 10 million deaths each year by 2020. According to the U.S. Center for Disease Control (CDC), tobacco use is the single leading preventable cause of death in the U.S., responsible for approximately 438,000 deaths each year. In addition, it is estimated that smoking results in an annual health-related economic cost of approximately $157 billion. The CDC estimates that, among the 45 million adult smokers in the U.S., 70% want to quit, but less than five percent of those who try to quit remain smoke-free after 12 months.

One reason it is difficult to quit smoking or to quit using tobacco products is addiction to the nicotine in cigarettes and other tobacco products. Nicotine is a small molecule that upon inhalation into the body quickly passes into the bloodstream and subsequently reaches the brain by crossing the blood-brain barrier. Once in the brain, the nicotine binds to nicotinic receptors, which results in the release of stimulants, such as dopamine, providing the smoker with a positive sensation, which leads to addiction.

There remains a need, therefore, for methods and kits for treating nicotine addiction.

### SUMMARY

In accordance with some embodiments, methods for treating nicotine addiction in a subject are provided that comprise (a) inducing a threshold level of anti-nicotine antibodies in a subject by administering one or more of (i) a nicotine immunogenic composition and (ii) a composition comprising anti-nicotine antibodies; and (b) administering a course of a nicotine receptor agonist or a nicotine receptor antagonist to the subject, wherein the threshold level of anti-nicotine antibodies is achieved in the subject by the time that the course of nicotine receptor agonist or nicotine receptor antagonist is completed. Similar methods for extending the duration of smoking abstinence in a subject who has quit smoking, increasing the likelihood of long-term abstinence from smoking, promoting smoking cessation in a subject, or preventing relapse of nicotine consumption following a period of nicotine abstinence in a subject also are provided,

In accordance with some embodiments, methods are provided that comprise (a) administering a course of a nicotine immunogenic composition to the subject; and (b) administering a course of a nicotine receptor agonist to the subject; wherein the relative timing of courses (a) and (b) overlap. In some embodiments, the relative timing of courses (a) and (b) is such that a first threshold level of anti-nicotine antibodies is achieved in the subject by the time that the course of the nicotine receptor agonist is completed. In accordance with some embodiments, anti-nicotine antibodies are used in conjunction with or instead of a nicotine immunogenic composition.

In any embodiments, the first threshold level of anti-nicotine antibodies may be selected from the group consisting of at least about 6 μg/ml, at least about 10 μg/ml, at least about 12 μg/ml, at least about 15 μg/ml, at least about 20 μg/ml, at least about 25 μg/ml, at least about 30 μg/ml, at least about 35 μg/ml, at least about 40 μg/ml, at least about 45 μg/ml, and at least about 50 μg/ml. In other embodiments, the first threshold level of anti-nicotine antibodies is at least about 1.5 to at least about 2.0 times the number of cigarettes smoked per day by the subject. In any embodiments where the subject has been administered a nicotine immunogenic composition, the threshold level of anti-nicotine antibodies may be directly correlated with the number of doses of a nicotine immunogenic composition that the subject has received and is selected from the group consisting of at least 10 μg/ml for a subject who has received up to two doses of a nicotine immunogenic composition; at least 25 μg/ml for a subject who has received three doses of a nicotine immunogenic composition; at least 50 μg/ml for a subject who has received four doses of a nicotine immunogenic composition, and at least 60 μg/ml for a subject who has received five or more doses of a nicotine immunogenic composition.

In some embodiments, the first threshold level of anti-nicotine antibodies is attained by about the 6th week through about the 12th week of the course the nicotine receptor agonist.

In accordance with any embodiments using a nicotine immunogenic composition, the nicotine immunogenic composition may be administered according to a course that comprises the administration of one to six doses of the nicotine immunogenic composition over about a six month period.

In accordance with any embodiments using a nicotine receptor agonist, the course of the nicotine receptor agonist may comprise the administration of at least a daily dose of the nicotine receptor agonist over a 12-week period.

In some embodiments, the course (a) of the nicotine immunogenic composition and/or anti-nicotine antibody composition is started before the course (b) of the nicotine receptor agonist is started. In some embodiments, a first dose of the course of the nicotine immunogenic composition is administered to the subject at least 2 weeks before a first dose of the course of the nicotine receptor agonist. In some embodiments, the course (a) of the nicotine immunogenic composition and or an anti-nicotine antibody composition is started substantially simultaneously with the course (b) of the nicotine receptor agonist. In some embodiments, the course (b) of the nicotine receptor agonist is started before the course (a) of the nicotine immunogenic composition and/or anti-nicotine antibody composition is started.

In some embodiments, the method comprises administering a composition comprising anti-nicotine antibodies substantially simultaneously with the course of nicotine receptor agonist or nicotine receptor antagonist. In other embodiments, the method comprises administering a composition comprising anti-nicotine antibodies near the end of the course of nicotine receptor agonist or nicotine receptor antagonist.

In some embodiments, the methods further comprise selecting a target quit date or counseling the subject to quit smoking about one week after the first dose of nicotine receptor agonist is administered. In some embodiments, a target quit date is selected, or the subject is counseled to quit smoking, when the subject's anti-nicotine antibody levels are at least a first threshold level.

In any embodiments using a nicotine immunogenic composition, the nicotine immunogenic composition may comprise a nicotine hapten conjugated to a suitable carrier protein. In some embodiments, the nicotine immunogenic composition comprises 3′aminomethylnicotine conjugated to a suitable carrier protein. In some embodiments, the carrier protein comprises recombinant exoprotein A.

In any embodiments using a nicotine receptor agonist, the nicotine receptor agonist may be a partial nicotine receptor agonist, such as varenicline.

In some embodiments, the methods comprise administering a course of a nicotine immunogenic composition to the subject, and administering a course of a nicotine receptor antagonist to the subject.

In any embodiments using a nicotine receptor antagonist, the threshold level of anti-nicotine antibodies may be achieved in the subject by the time that the efficacy of the nicotine receptor antagonist is diminishing. In any embodiments using a nicotine receptor antagonist, the nicotine receptor antagonist may comprise bupropion.

In accordance with some embodiments, there are provided methods for extending the duration of smoking abstinence in a subject who has quit smoking, increasing the likelihood of long-term abstinence from smoking in a subject, promoting smoking cessation in a subject, or preventing relapse of nicotine consumption following a period of nicotine abstinence in a subject, comprising (a) administering a course of a nicotine immunogenic composition and/or an anti-nicotine antibody composition to the subject; and (b) administering a course of a nicotine receptor agonist to the subject; wherein the relative timing of courses (a) and (b) overlap.

In accordance with some embodiments, there are provided methods for treating nicotine addiction in a subject comprising: (a) administering a course of a nicotine immunogenic composition and/or an anti-nicotine antibody composition to the subject; and (b) administering a course of a nicotine receptor antagonist to the subject; wherein the relative timing of courses (a) and (b) overlap. In accordance with some embodiments, the relative timing of courses (a) and (b) is such that a first threshold level of anti-nicotine antibodies is achieved in the subject by the time that the course of the nicotine receptor antagonist is completed. In accordance with some embodiments, the relative timing of courses (a) and (b) is such that a first threshold level of anti-nicotine antibodies is achieved in the subject by the time that the efficacy of the nicotine receptor antagonist is diminishing. In some embodiments, the nicotine receptor antagonist comprises a partial nicotine receptor antagonist, such as bupropion.

In accordance with other embodiments, there are provided methods for treating nicotine addiction in a subject comprising inducing a threshold level of anti-nicotine antibodies in a subject by administering (a) a nicotine immunogenic composition and (b) a composition comprising anti-nicotine antibodies. In some embodiments, these methods comprise (a) administering a nicotine immunogenic composition and measuring the subject's serum anti-nicotine antibody level, and, (b) if the measured serum anti-nicotine antibody level is below a threshold level, administering anti-nicotine antibodies. In further embodiments, the methods may further comprise administering a nicotine receptor agonist and/or a nicotine receptor antagonist.

In accordance with other embodiments, there are provided methods for treating nicotine addiction in a subject comprising: (a) administering one or more of a first nicotine immunogenic composition and a first anti-nicotine antibody composition; (b) measuring the level of anti-nicotine antibodies in serum from said subject; (c) if the measured serum anti-nicotine antibody level is below a threshold level, administering one or more of: (i) a second nicotine immunogenic composition; (ii) a second anti-nicotine antibody composition; and (iii) a nicotine receptor agonist and/or antagonist. In some embodiments, step (a) further comprises administering a nicotine receptor agonist and/or nicotine receptor antagonist. In some embodiments, methods further comprise, after step (c), (d) measuring the level of anti-nicotine antibodies in serum from said subject; (e) if the measured serum anti-nicotine antibody level is below a threshold level, administering one or more of (i) a nicotine immunogenic composition; (ii) anti-nicotine antibodies; and (iii) a nicotine receptor agonist and/or antagonist.

In accordance with some embodiments, there are provided kits for treating nicotine addiction in a subject comprising: (a) at least one dose of one or more of a nicotine immunogenic composition and an anti-nicotine antibody composition; (b) at least one dose of a nicotine receptor agonist and/or nicotine receptor antagonist; and (c) instructions for administering to the subject the dose(s) of the nicotine immunogenic composition and/or anti-nicotine antibody composition and the dose(s) of the nicotine receptor agonist and/or nicotine receptor antagonist in overlapping courses such that a threshold level of anti-nicotine antibodies is achieved in the subject by the time that the course of nicotine receptor agonist or nicotine receptor antagonist is completed. In some embodiments, the kits comprise at least one dose of a nicotine immunogenic composition. In some embodiments, the kits comprise at least one dose of an anti-nicotine antibody composition. In some embodiments, the kits further comprise an agent for detecting anti-nicotine antibodies in serum obtained from a subject.

In accordance with some embodiments, there are provided kits for treating nicotine addiction in a subject comprising: (a) at least one dose of a nicotine immunogenic composition and/or an anti-nicotine antibody composition; (b) at least one dose of a nicotine receptor agonist and/or nicotine receptor antagonist; and (c) instructions for administering to the subject the dose(s) of the nicotine immunogenic composition and/or the anti-nicotine antibody composition and the dose(s) of the nicotine receptor agonist and/or nicotine receptor antagonist in overlapping courses.

In accordance with some embodiments, there are provided kits for treating nicotine addiction in a subject comprising: (a) at least one dose of a nicotine immunogenic composition and/or an anti-nicotine antibody composition; (b) instructions for administering to the subject the dose(s) of the nicotine immunogenic composition and/or the anti-nicotine antibody composition in conjunction with a course of a nicotine receptor agonist and/or nicotine receptor antagonist.

In accordance with some embodiments, there are provided kits for treating nicotine addiction in a subject comprising: (a) at least one dose of a nicotine receptor agonist and/or nicotine receptor antagonist; and (b) instructions for administering to the subject the dose(s) of the nicotine receptor agonist and/or nicotine receptor antagonist in conjunction with a course of a nicotine immunogenic composition and/or an anti-nicotine antibody composition.

In accordance with other embodiments there are provided kits for treating nicotine addiction in a subject comprising (a) at least one dose of a nicotine immunogenic composition; (b) at least one dose of an anti-nicotine antibody composition; and (c) instructions for administering to the subject the dose(s) of the nicotine immunogenic composition and anti-nicotine antibody composition to achieve a threshold serum anti-nicotine antibody level.

In some embodiments of the kits described herein, the instructions indicate that the relative timing of administration to the subject of the nicotine immunogenic composition and/or an anti-nicotine antibody composition and nicotine receptor agonist and/or nicotine receptor antagonist should be such that a first threshold level of anti-nicotine antibodies is achieved in the subject by the time that the course of the nicotine receptor agonist and/or nicotine receptor antagonist is completed.

In accordance with other embodiments, combinations are provided comprising (a) one or more of (i) a nicotine immunogenic composition and (ii) an anti-nicotine antibody composition, and (b) a nicotine receptor agonist or a nicotine receptor antagonist, for use in the treatment of nicotine addiction in a subject, wherein the nicotine immunogenic composition and/or anti-nicotine antibody composition are administered to induce a threshold level of anti-nicotine antibodies in the subject, and a course of nicotine receptor agonist or nicotine receptor antagonist is administered simultaneously or sequentially with the nicotine immunogenic composition and/or anti-nicotine antibody composition, wherein the threshold level of anti-nicotine antibodies is achieved in the subject by the time that the course of nicotine receptor agonist or nicotine receptor antagonist is completed. Specific embodiments of such combinations include those parallel to the methods and kits described herein.

In accordance with other embodiments, combinations are provided comprising (a) one or more of (i) a nicotine immunogenic composition and (ii) an anti-nicotine antibody composition, and (b) a nicotine receptor agonist or a nicotine receptor antagonist, for use in extending the duration of smoking abstinence in a subject who has quit smoking, increasing the likelihood of long-term abstinence from smoking by a subject, promoting smoking cessation in a subject, or preventing relapse of nicotine consumption following a period of nicotine abstinence in a subject, wherein the nicotine immunogenic composition and/or anti-nicotine antibody composition are administered to induce a threshold level of anti-nicotine antibodies in the subject, and a course of nicotine receptor agonist or nicotine receptor antagonist is administered simultaneously or sequentially with the nicotine immunogenic composition and/or anti-nicotine antibody composition, wherein the threshold level of anti-nicotine antibodies is achieved in the subject by the time that the course of nicotine receptor agonist or nicotine receptor antagonist is completed. Specific embodiments of such combinations include those parallel to the methods and kits described herein.

In accordance with other embodiments, combinations are provided comprising a nicotine immunogenic composition and an anti-nicotine antibody composition, for use in the treatment of nicotine addiction in a subject, wherein the nicotine immunogenic composition is administered to the subject, the subject's serum anti-nicotine antibody level is measured and, if the measured serum anti-nicotine antibody level is below a threshold level, the anti-nicotine antibody composition is administered to the subject. Specific embodiments of such combinations include those parallel to the methods and kits described herein.

In accordance with other embodiments, combinations are provided comprising (a) one or more of a first nicotine immunogenic composition and a first anti-nicotine antibody composition and (b) a second nicotine immunogenic composition, a second anti-nicotine antibody composition, and a nicotine receptor agonist and/or antagonist, for use in the treatment of nicotine addiction, wherein the first nicotine immunogenic composition and/or first anti-nicotine antibody composition is administered to the subject, the subject's serum anti-nicotine antibody level is measured and, if the measured serum anti-nicotine antibody level is below a threshold level, the second nicotine immunogenic composition, second anti-nicotine antibody composition, or second nicotine receptor agonist and/or antagonist, is administered to the subject. Specific embodiments of such combinations include those parallel to the methods and kits described herein.

In accordance with other embodiments, there is provided the use of (a) one or more of (i) a nicotine immunogenic composition and (ii) an anti-nicotine antibody composition, and (b) a nicotine receptor agonist or a nicotine receptor antagonist, in the manufacture of a medicament for the treatment of nicotine addiction in a subject, wherein the nicotine immunogenic composition and/or anti-nicotine antibody composition are administered to induce a threshold level of anti-nicotine antibodies in the subject, and a course of nicotine receptor agonist or nicotine receptor antagonist is administered simultaneously or sequentially with the nicotine immunogenic composition and/or anti-nicotine antibody composition, wherein the threshold level of anti-nicotine antibodies is achieved in the subject by the time that the course of nicotine receptor agonist or nicotine receptor antagonist is completed. Specific embodiments of such uses include those parallel to the methods and kits described herein.

In accordance with other embodiments, there is provided the use of (a) one or more of (i) a nicotine immunogenic composition and (ii) an anti-nicotine antibody composition, and (b) a nicotine receptor agonist or a nicotine receptor antagonist, in the manufacture of a medicament for extending the duration of smoking abstinence in a subject who has quit smoking, increasing the likelihood of long-term abstinence from smoking by a subject, promoting smoking cessation in a subject, or preventing relapse of nicotine consumption following a period of nicotine abstinence in a subject, wherein the nicotine immunogenic composition and/or anti-nicotine antibody composition are administered to induce a threshold level of anti-nicotine antibodies in the subject, and a course of nicotine receptor agonist or nicotine receptor antagonist is administered simultaneously or sequentially with the nicotine immunogenic composition and/or anti-nicotine antibody composition, wherein the threshold level of anti-nicotine antibodies is achieved in the subject by the time that the course of nicotine receptor agonist or nicotine receptor antagonist is completed. Specific embodiments of such uses include those parallel to the methods and kits described herein.

In accordance with other embodiments, there is provided the use of a nicotine immunogenic composition and an anti-nicotine antibody composition, in the manufacture of a medicament for the treatment of nicotine addiction in a subject, wherein the nicotine immunogenic composition is administered to the subject, the subject's serum anti-nicotine antibody level is measured and, if the measured serum anti-nicotine antibody level is below a threshold level, the anti-nicotine antibody composition is administered to the subject. Specific embodiments of such uses include those parallel to the methods and kits described herein.

In accordance with other embodiments, there is provided the use of (a) one or more of a first nicotine immunogenic composition and a first anti-nicotine antibody composition and (b) a second nicotine immunogenic composition, a second anti-nicotine antibody composition, and a nicotine receptor agonist and/or antagonist, in the manufacture of a medicament for the treatment of nicotine addiction, wherein the first nicotine immunogenic composition and/or first anti-nicotine antibody composition is administered to the subject, the subject's serum anti-nicotine antibody level is measured and, if the measured serum anti-nicotine antibody level is below a threshold level, the second nicotine immunogenic composition, second anti-nicotine antibody composition, or second nicotine receptor agonist and/or antagonist, is administered to the subject. Specific embodiments of such uses include those parallel to the methods and kits described herein.

### BRIEF DESCRIPTION OF THE DRAWINGS

FIG. 1 shows the geometric mean concentration (GMC) of subject serum antibody levels (μg/ml) between 0 and 52 weeks of the subjects in a clinical study that evaluated two different vaccination schedules at two different doses each. (Δ: 200 μg/Schedule 1; □: 400 μg/Schedule 1; ▴: 200 μg/Schedule 2; ▪: 400 μg/Schedule 2).

FIG. 2 illustrates the protocol of a method as described herein.

### DETAILED DESCRIPTION

Disclosed herein are methods and kits useful for treating nicotine addiction and nicotine-addiction related disorders, promoting smoking cessation, extending the duration of smoking abstinence in a subject who has quit smoking, increasing the likelihood of long-term abstinence, preventing relapse (e.g., reducing the likelihood of a relapse) of nicotine consumption following a period of nicotine abstinence, and/or rescuing a subject from relapse (including relapse in a subject who initially achieved abstinence while being treated in accordance with one of the methods described herein). The methods and kits described herein include a combination of a nicotine immunogenic composition (e.g., a nicotine vaccine) and/or anti-nicotine antibodies and/or a nicotine receptor agonist and/or a nicotine receptor antagonist.

One exemplary vaccine is a nicotine-hapten carrier conjugate based vaccine being developed by Nabi Biopharmaceuticals under the name NicVAX®. The NicVAX® nicotine-hapten carrier conjugate (and vaccines and immunogenic compositions comprising it) is described, for example, in U.S. Pat. No. 6,232,082, the entire contents of which are incorporated herein by reference. Other suitable vaccines are described below.

Exemplary anti-nicotine antibodies and compositions comprising them are described, for example, in U.S. Pat. No. 6,232,082. In accordance with some embodiments, the anti-nicotine antibodies are monoclonal antibodies, polyclonal antibodies, single chain antibodies, recombinant antibodies or combinations thereof, that specifically bind nicotine. Additionally or alternatively, antibody fragments that bind nicotine are used. In some embodiments the antibodies or fragments are from a human or are fully or partially humanized. Other suitable antibodies and antibody compositions are described below.

In some embodiments, the nicotine receptor agonist is a partial receptor agonist. One exemplary partial receptor agonist is varenicline (presently marketed as CHANTIX® or CHAMPIX). In other embodiments, the nicotine receptor antagonist is a non-competitive antagonist. One exemplary non-competitive antagonist is bupropion (presently marketed as ZYBAN®).

As used herein, the term “nicotine receptor agonist” does not include nicotine.

In some embodiments, one or more other smoking-cessation agents are used as an alternative to, or in addition to, a nicotine receptor agonist and/or antagonist. Examples of such other smoking-cessation agents include, but are not limited to, one or more of a nicotinic cholinergic antagonist (such as mecylamine), monoamine oxidase inhibitors, glycine antagonists, opiate antagonists and agonists, dopamine D3 antagonists, nicotinic ligands, dopamine uptake inhibitors, cannabinoid receptor 1 antagonists, inhibitors of enzymes involved in nicotine and/or cotinine metabolism, including cytochrome P450 enzymes (e.g., cytochrome p450 2A6-CYP2A6), aldehyde oxidase, flavin-containing monooxygenase 3, amine N-methyltransferase, and UDP-glucuronosyltransferases.

In accordance with some embodiments, there are provided methods for treating nicotine addiction, promoting smoking cessation, extending the duration of smoking abstinence in a subject who has quit smoking, increasing the likelihood of long-term abstinence, preventing relapse (e.g., reducing the likelihood of a relapse) of nicotine consumption following a period of nicotine abstinence, and/or rescuing a subject from relapse (including relapse in a subject who initially achieved abstinence while being treated in accordance with one of the methods described herein), comprising (a) administering a course of a nicotine immunogenic composition to the subject; and (b) administering a course of a nicotine receptor agonist or antagonist to the subject, wherein the relative timing of courses (a) and (b) overlap. Thus, in some embodiments, the methods, compositions and kits described herein employ a combination of a nicotine immunogenic composition and a nicotine receptor agonist and/or antagonist. In some embodiments, anti-nicotine antibodies are administered in addition to or as an alternative to the nicotine immunogenic composition. The present inventors believe that the combinations described herein achieve unexpected benefits in the improved treatment of nicotine addiction and promotion of smoking cessation. This improved effect may be due in part to the fact that each component impacts different aspects of nicotine consumption and/or addiction. For example, nicotine receptor agonists and antagonists act centrally, at the site of nicotine receptors in the brain, while anti-nicotine antibodies (administered directly or induced by a nicotine immunogenic composition) act peripherally, for example, binding circulating nicotine. The anti-nicotine antibodies (administered directly or induced by a nicotine immunogenic composition) reduce the amount of nicotine that reaches the brain. This may be correlated with, for example, reduced cravings for nicotine. Moreover, the use of nicotine immunogenic compositions and/or anti-nicotine antibodies in combination with a nicotine receptor agonist and/or antagonist may reduce the dose of nicotine receptor agonist and/or antagonist required to be effective. In addition, the improved effect may be due to the relative timing of administration of the components, with each component complementing and potentiating the effects of the other.

The nicotine immunogenic composition induces the production of anti-nicotine antibodies in the subject. The general theory behind nicotine vaccines is that they induce nicotine-specific antibodies that bind nicotine and reduce its distribution to the brain, blocking nicotine drug effects, including those responsible for nicotine addiction. See, e.g., Hatsukani et al., Clin. Pharm. & Ther. 78: 456-67 (2005). The same effect can be achieved by the direct administration of anti-nicotine antibodies. Previous studies with NicVAX® reveal that the efficacy of a nicotine vaccine is apparent once anti-nicotine antibody levels have reached a first minimum threshold level (which typically occurs four weeks or longer after the first dose of NicVAX®), and that their impact can endure for as long as anti-nicotine antibody levels are maintained at a second minimum threshold level (which typically may be one year or longer after a course of four or five administrations of NicVAX®), as discussed in more detail below.

Nicotine receptor agonists and antagonists act at the nicotine receptors in the brain by preventing effective binding of nicotine to nicotine receptors, thereby diminishing the positive feelings derived from nicotine consumption. Previous studies with varenicline indicate that its efficacy generally is apparent one week after the first dose is administered and is maintained throughout the course of administration, which typically is 12 weeks. Previous studies with bupropion indicate that its efficacy generally is apparent one week after the first dose is administered and may diminish after about six or seven weeks of administration, even though administration is ongoing. Repeat courses of treatment of these drugs often are followed, although side-effects associated with these drugs make this undesirable.

In accordance with the methods described herein, the relative timing of the course of the nicotine immunogenic composition and/or anti-nicotine antibodies and the course of the nicotine receptor agonist and/or antagonist is such that a first threshold level of anti-nicotine antibodies is achieved in the subject (e.g., induced by the nicotine immunogenic composition and/or by administration of anti-nicotine antibodies) by the time that the course of the nicotine receptor agonist and/or antagonist is completed and/or by the time that the efficacy of the nicotine receptor agonist or antagonist is diminishing or has diminished. For example, the course of the nicotine immunogenic composition and/or anti-nicotine antibodies may be started before the course of the nicotine receptor agonist and/or antagonist is started, or may be started substantially simultaneously with the course of the nicotine receptor agonist and/or antagonist. In embodiments where the nicotine immunogenic composition induces anti-nicotine antibodies in a relatively short period of time compared to the duration of the course/efficacy of the nicotine receptor agonist and/or antagonist, or when anti-nicotine antibodies are administered, the course of the nicotine immunogenic composition and/or anti-nicotine antibodies may be started after the course of the nicotine receptor agonist and/or antagonist is started. In accordance with each of these embodiments, the relative timing of the courses of the nicotine immunogenic composition and/or anti-nicotine antibodies and nicotine receptor agonist and/or antagonist overlap or are administered such that a first threshold level of anti-nicotine antibodies is achieved in the subject (as discussed in more detail below) before the course of the nicotine receptor agonist and/or antagonist is completed and/or before the efficacy of the nicotine receptor agonist and/or antagonist diminishes to ineffective levels.

In accordance with some embodiments, there are provided methods for treating nicotine addiction, promoting smoking cessation, extending the duration of smoking abstinence in a subject who has quit smoking, increasing the likelihood of long-term abstinence, preventing relapse (e.g., reducing the likelihood of a relapse) of nicotine consumption following a period of nicotine abstinence, and/or rescuing a subject from relapse (including relapse in a subject who initially achieved abstinence while being treated in accordance with one of the methods described herein), comprising (a) administering a course of a nicotine immunogenic composition to the subject; and (b) administering anti-nicotine antibodies to the subject. The relative timing of administering the nicotine immunogenic composition and anti-nicotine antibodies can be tailored to achieve a threshold level of serum antibodies at a given time. Thus, in some embodiments, the methods, compositions and kits described herein employ a combination of a nicotine immunogenic composition and a nicotine antibody composition. In some embodiments, anti-nicotine antibodies are administered in addition to or as an alternative to the nicotine immunogenic composition, at the same time or at a different time as the nicotine immunogenic composition. such as one or more weeks after a dose of nicotine immunogenic composition.

Embodiments using anti-nicotine antibodies achieve unexpected benefits in the improved treatment of nicotine addiction and promotion of smoking cessation, This improved effect may be due in part to being able to achieve and/or maintain a threshold level of anti-nicotine antibodies in the subject with more certainty than by the use of a nicotine immunogenic composition alone. Moreover, since the administration of anti-nicotine antibodies results in a nearly immediate increase of serum antibody levels, a subject whose anti-nicotine antibody levels are below a threshold level can be administered anti-nicotine antibodies to achieve the threshold level nearly instantaneously. Thus, for example, while administration of a nicotine immunogenic composition typically results in a gradual increase in serum anti-nicotine antibody levels, the administration of anti-nicotine antibodies can be tailored to achieve a threshold serum antibody level more quickly. On the other hand, the administration of anti-nicotine antibodies can be tailored to mimic the gradual increase that would be achieved with a nicotine immunogenic composition, which may offer the further advantage of reducing nicotine withdrawal symptoms and/or cravings. Thus, the specific course of administration of anti-nicotine antibodies can be tailored to meet a specific subject's (or target patient population's) needs, such as by being based on whether it is important that the subject be able to cease nicotine consumption as soon as possible (such as may be the case, for example, for cancer patients) or whether a more gradual approach can be taken (such as, for example, for subjects for whom withdrawal symptoms are a significant concern). Thus in some embodiments, anti-nicotine antibodies are achieved in a subject by administration of a nicotine immunogenic composition, administration of anti-nicotine antibodies, or both.

In accordance with the methods described herein, subjects may quit smoking earlier than subjects treated with only one anti-nicotine agent alone (e.g., with only a nicotine immunogenic composition or only anti-nicotine antibodies or only a nicotine receptor agonist or antagonist), and a subject's likelihood of quitting smoking may increase as compared to the likelihood with treatment with only one component of the combination treatment (e.g., as compared to treatment with a nicotine immunogenic composition but not with anti-nicotine antibodies or a nicotine receptor agonist or antagonist, or treatment with a nicotine receptor agonist or antagonist but not a nicotine immunogenic composition or anti-nicotine antibodies, or treatment with anti-nicotine antibodies and not with a nicotine agonist and/or antagonist or a nicotine immunogenic composition). Moreover, the methods described herein may extend the duration of smoking abstinence in a subject who has quit smoking (including a subject who has quit smoking while being treated in accordance with one of the methods), increase the likelihood of long-term abstinence, and/or prevent relapse (e.g., reduce the likelihood of a relapse) of nicotine consumption following a period of nicotine abstinence (including abstinence in a subject who has achieved abstinence while being treated in accordance with one of the methods), and/or rescue a subject from relapse (including relapse in a subject who initially achieved abstinence while being treated in accordance with one of the methods).

Before describing the invention in further detail, the following definitions are provided.

As used herein, the singular forms “a,” “an,” and “the” designate both the singular and the plural, unless expressly stated to designate the singular only.

The term “about” and the use of ranges in general, whether or not qualified by the term about, means that the number comprehended is not limited to the exact number set forth herein, and is intended to refer to ranges substantially within the quoted range while not departing from the scope of the invention. As used herein, “about” will be understood by persons of ordinary skill in the art and will vary to some extent on the context in which it is used. If there are uses of the term which are not clear to persons of ordinary skill in the art given the context in which it is used, “about” will mean up to plus or minus 10% of the particular term.

As used herein a “subject” or a “patient” are used interchangeably and refer to someone who desires to cease nicotine consumption or quit smoking, including someone in need of smoking cessation treatment, nicotine addiction treatment, initiation or extension of abstinence from nicotine, and/or prevention of or rescue from relapse of nicotine consumption. A subject or patient may be a human subject who smokes cigarettes or uses other tobacco products or chews tobacco, or uses other nicotine products. Such a subject may or may not be physically addicted to nicotine and/or psychologically addicted to smoking cigarettes or using other tobacco or other nicotine products. Typical subjects smoke or use tobacco or other nicotine products daily, such as smoking at least 1 cigarette a day, or more, such as at least about 5, at least about 10, at least about 15, at least about 20, or more, cigarettes per day, including fewer than 10, 10-20, 20-30, 30-40, or 40 or more (or the equivalent use of other tobacco or nicotine products). Other nicotine products include, but are not limited to chewing tobacco, pipes, cigars, electronic cigarettes, and other nicotine delivery devices.

As used herein, a “nicotine immunogenic composition” refers to a composition that induces anti-nicotine antibodies in the subject or elevates the levels of anti-nicotine antibodies in the subject, such as a nicotine vaccine. Such a composition or vaccine generally is in a form that is capable of being administered to a subject, and may comprises a conventional saline or buffered aqueous solution medium in addition to the antigenic moiety. Optionally, the composition or vaccine additionally includes an adjuvant which can be present in either a minor or major proportion relative to the antigen. A “nicotine immunogenic composition” can include a combination of one or more nicotine vaccines or nicotine immunogenic compositions (used independently, concurrently, or in combination), and includes multivalent nicotine vaccines and nicotine immunogenic compositions that include two or more nicotine antigens, for example, that may comprise the same or different nicotine hapten, the same or different immunogenic carrier, or the same or different nicotine hapten-carrier conjugate (such as by being conjugated by a different linker or at a different site).

As used herein “serum” includes blood or plasma. A sample of blood from the subject can be used to assess serum antibody levels. Additionally or alternatively, saliva from the subject can be used to assess secreted antibody levels. For convenience, serum antibody levels are discussed, but it should be understood that antibody levels could be determined with reference to secreted antibody levels. Moreover, the practitioner can determine corresponding secreted antibody levels using routine methodologies.

As used herein, the term “effective amount” refers to an amount necessary or sufficient to realize a desired biologic effect. An effective amount of a composition is the amount that achieves this selected result, and such an amount could be determined as a matter of routine by a person skilled in the art. The term is also synonymous with “sufficient amount.” The effective amount for any particular application can vary depending on such factors as the disease or condition being treated, the particular composition being administered, the size of the subject, and/or the severity of the disease or condition. One of ordinary skill in the art can empirically determine the effective amount of a particular composition without necessitating undue experimentation. It should be understood that an effective amount may not, in fact, realize a desired biologic effect in a particular subject, although the amount has been determined to be an effective amount based on one or more studies in other subjects.

### I. NICOTINE IMMUNOGENIC COMPOSITIONS

Nicotine immunogenic compositions (e.g., vaccines) have been disclosed in the art as smoking cessation aids. Typically such compositions include a nicotine-carrier conjugate that is administered to induce anti-nicotine antibodies. A “nicotine-carrier conjugate” designates a compound that comprises a nicotine hapten (e.g., a nicotine molecule or a nicotine derivative) linked to an immunogenic molecule, or carrier. Such a linkage may be a covalent linkage, and may be direct or via a linker or linking moiety. The nicotine-carrier conjugate is capable of inducing anti-nicotine antibodies (e.g., antibodies that specifically bind nicotine). Examples of such conjugates, and methods for their preparation, are well known in the art. See, for example, U.S. Pat. No. 6,232,082 (Ennifar), U.S. App. 2007/0129551 A1 (Ennifar), U.S. Pat. No. 5,876,727 (Swain) and U.S. Pat. No. 6,932,971 (Bachmann) (describing nicotine-virus like particle conjugates). In some embodiments, the nicotine-carrier conjugates include 3′ aminomethylnicotine, for example, 3′aminomethylnicotine conjugated to recombinant exoprotein A. One non-limiting nicotine vaccine is the NicVAX® product made by Nabi Biopharmaceuticals (Rockville, Md.).

The nicotine immunogenic compositions described herein may contain at least one adjuvant. The adjuvant used in accordance with the present invention is selected so as to not inhibit the effect of the carrier molecule. Exemplary adjuvants include those that are physiologically acceptable to humans, including alum, QS-21, saponin and monophosphoryl lipid A.

The nicotine immunogenic compositions described herein may optionally contain one or more pharmaceutically acceptable excipients. Exemplary excipients include, but are not limited to sterile water, salt solutions such as saline, sodium phosphate, sodium chloride, alcohol, gum arabic, vegetable oils, benzyl alcohols, polyethylene glycol, gelatin, mannitol, carbohydrates, magnesium stearate, viscous paraffin, fatty acid esters, hydroxyl methyl cellulose and buffers. Other excipients suitable for use in accordance with the present invention are known in the art. In addition to the usual pharmaceutically acceptable excipients, the composition may contain optional components to ensure purity, enhance the bioavailability and/or increase penetration.

The formulation of the nicotine-carrier conjugate may optionally contain at least one auxiliary agent, including, but not limited to dispersion media, coatings, microsphere, liposomes, microcapsules, lipids, surfactants, lubricants, preservatives and stabilizers. Other auxiliary agents suitable for use in the vaccine formulation are known in the art.

Pharmaceutical compositions including the nicotine-carrier conjugate may contain additional components in order to protect the composition from infestation with, and growth of, microorganisms. In one embodiment, the composition is manufactured in the form of a lyophilized powder which is to be reconstituted by a pharmaceutically acceptable diluent just prior to administration. Methods of preparing sterile injectable solutions are well known to the skilled artisan and include but are not limited to drying, freeze-drying, and spin drying. These techniques yield a powder of the active ingredient with any additional excipient incorporated therein.

In some embodiments, the nicotine immunogenic composition is an extended release formulation. Such embodiments may be particularly useful when the course of nicotine immunogenic composition consists of a single dose of nicotine immunogenic composition, or when the course consists of only one, two, three or four doses of nicotine immunogenic composition.

The immunogenic compositions can be administered by a variety of means, including via intranasal, intrathecal, oral, dermal, subcutaneous, or intravenous modes of administration. When the composition containing the hapten carrier conjugate is to be used for injection, it is typical to solubilize the hapten carrier conjugate in an aqueous saline solution at a pharmaceutically acceptable pH. However, it is possible to use an injectable suspension of the hapten carrier conjugate.

A nicotine immunogenic composition can be administered in a single dose or in multiple doses. For example, following initial administration of a dose of nicotine immunogenic composition, a subsequent administration of one or more “boosters” may follow. Such a booster will increase anti-nicotine antibody levels. However, a single dose of the nicotine carrier conjugate is also specifically contemplated. As used herein a “course” of nicotine immunogenic composition includes any number of doses effective to induce anti-nicotine antibodies, including a singe dose or multiple doses, and includes courses using only one nicotine vaccine or nicotine immunogenic composition and courses using two or more different nicotine vaccines or nicotine immunogenic compositions, and courses using one or more multivalent nicotine vaccines or nicotine immunogenic compositions.

As noted above, previous studies with NicVAX® reveals that the efficacy of a nicotine immunogenic composition in the context of, for example, smoking cessation, is apparent once anti-nicotine antibody levels have reached a first minimum threshold level (which typically occurs about four weeks or longer after the first dose of NicVAX®), and that their impact can endure for as long as anti-nicotine antibody levels are maintained at a second minimum threshold level (which typically may be about one year or longer after a course of four or five administrations of NicVAX®). This is described in more detail in U.S. patent application Ser. No. 12/481,420, filed Jun. 9, 2009, the entire contents of which are incorporated herein by reference in their entirety, and international patent application PCT/US09/47679, filed Jun. 9, 2009, the entire contents of which are incorporated herein by reference in their entirety. For example, it has been determined that when serum or secreted anti-nicotine antibody levels reach a threshold level, the chance for a successful quit attempt is significantly increased. While not wanting to be bound by any theory, it is believed that the greater the serum or secreted anti-nicotine antibody level, the greater the chance of a successful quit attempt.

Exemplary first minimum threshold serum antibody levels include at least about 6 μg/ml, at least about 10 μg/ml, at least about 12 μg/ml, at least about 15 μg/ml, at least about 20 μg/ml, at least about 25 μg/ml, at least about 30 μg/ml, at least about 35 μg/ml, at least about 40 μg/ml, at least about 45 μg/ml, or, at least about 50 μg/ml, such as at least 6 μg/ml, at least 10 μg/ml, at least 12 μg/ml, at least 15 μg/ml, at least 20 μg/ml, at least 25 μg/ml, at least 30 μg/ml, at least 35 μg/ml, at least 40 μg/ml, at least 45 μg/ml, or at least 50 μg/ml. In other embodiments, the first minimum threshold serum anti-nicotine antibody level (in μg/ml) is from at least about 1.5 to at least about 2.0 times the number of cigarettes smoked per day by the subject, such as being 1.5 to 2.0 times the number of cigarettes smoked per day. In other embodiments, the first minimum threshold level is directly correlated with the number of doses of a nicotine immunogenic composition that the subject has received prior to the measuring of the level of anti-nicotine antibodies. For instance, the first specified threshold anti-nicotine antibody level can be selected from at least about 10 μg/ml for up to two prior doses (such as from at least 10 to at least 25 μg/ml), at least about 25 μg/ml for three prior doses (such as from at least 25 to at least 50 μg/ml), at least about 50 μg/ml for four prior doses (such as from at least 50 to at least 75 μg/ml), and at least about 60 μg/ml for five or more prior doses (such as from at least 60 to at least 75 or at least 100 μg/ml). The practitioner readily can determine corresponding secreted antibody levels using routine methodologies.

As those skilled in the art will understand, the level of antibodies induced by a given nicotine immunogenic composition and the time necessary to induce a certain threshold antibody level will vary from subject to subject and nicotine immunogenic composition to nicotine immunogenic composition. For example, the potency of a nicotine immunogenic composition may depend on the specific hapten, the specific carrier, the specific hapten-carrier conjugate, whether an adjuvant is present and, if so, its potency, and the route of administration. Thus, the dosage guidelines provided herein are exemplary only, and suitable doses of a given nicotine immunogenic composition can be determined by the skilled practitioner.

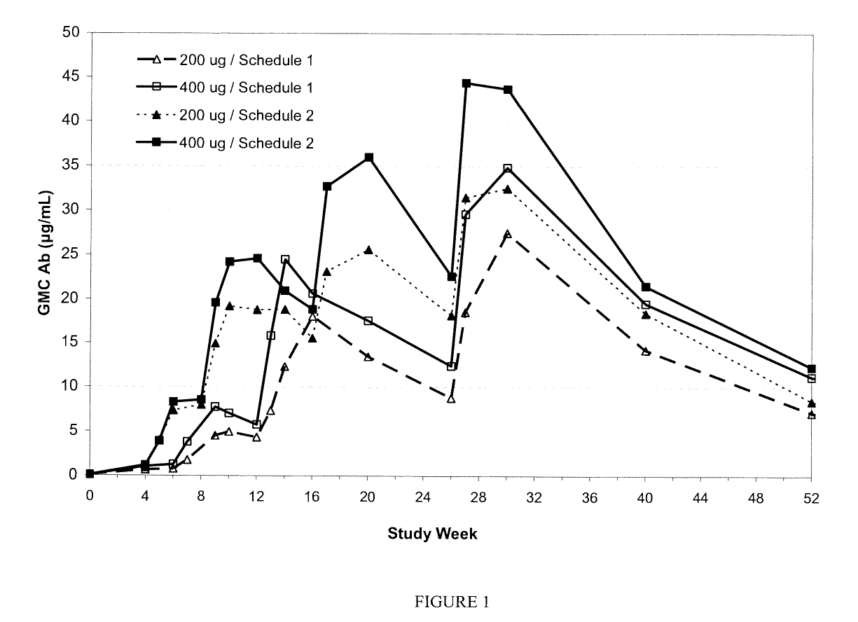

FIG. 1 shows the geometric mean concentration (GMC) of subject serum antibody levels (μg/ml) between 0 and 52 weeks of the subjects in a randomized, double-blind, clinical study that was conducted with 301 human subjects. All subjects were heavy smokers—the average number of cigarettes smoked per day was 24, with no subject smoking less than 15 cigarettes per day. 201 subjects were treated with NicVAX® and 100 received placebo treatment (phosphate buffered saline and alum). Two dosing schedules and two dosage levels of NicVAX® were tested. Under Schedule 1, 50 subjects were dosed intravenously with 400 μg or 200 μg of NicVAX® (and alum adjuvant) at 0, 6, 12 and 26 weeks. Under Schedule 2, 51 and 50 subjects were dosed with 400 μg or 200 μg of NicVAX® (and alum adjuvant) at 0, 4, 8, 16 and 26 weeks. For each dosing schedule, 50 placebo subjects received PBS and alum. As shown in FIG. 1, schedule 2 achieves higher serum antibody levels earlier, and schedule 2 serum antibody levels remain higher than the schedule 1 antibody levels throughout the 52-week study period (Δ: 200 μg/Schedule 1; □: 400 μg/Schedule 1; ▴: 200 μg/Schedule 2; ▪: 400 μg/Schedule 2). As discussed in more detail in U.S. patent application Ser. No. 12/481,420 and PCT/US09/47679, the clinical trial results indicate that subjects with higher concentrations of serum anti-nicotine antibodies had better quit rates and longer continuous abstinence rates than subjects with lower concentrations of the anti-nicotine antibodies and the placebo control group. The tables below show the numbers and percentages of subjects who achieved total abstinence (“Continuous Abstinence Rate”) by the 6 month, 9 month and 12 month time points by dose group, and the numbers and percentages of twelve-month continuous abstinence (smoking cessation) based on treatment group and subject serum antibody levels (high versus low serum antibody levels).

TABLE IA
Six, Nine & Twelve Month Continuous
Abstinence Rates By NicVAX ® Schedule

| | 6-Month | 9-Month | 12-Month |
|---|---|---|---|
| | 20-Week | 34-Week | 44-Week |
| NicVAX ® | CAR | CAR | CAR |
| Schedule 2 | 18% | 18% | 16% |
| 400 μg | (n = 9/51) | (n = 9/51) | (n = 8/51) |
| | p = 0.015 | p = 0.016 | p = 0.038 |
| Schedule 2 | 14% | 14% | 14% |
| 200 μg | (n = 7/50) | (n = 7/50) | (n = 7/50) |
| | p = 0.054 | p = 0.053 | p = 0.056 |
| Schedule 1 | 6% | 6% | 6% |
| 400 μg | (n = 3/50) | (n = 3/50) | (n = 3/50) |
| | p = 0.87 | p = 0.92 | p = 0.96 |
| Schedule 1 | 8% | 6% | 6% |
| 200 μg | (n = 4/50) | (n = 3/50) | (n = 3/50) |
| | p = 0.84 | p = 0.88 | p = 0.88 |
| Placebo | 6% | 6% | 6% |
| | (6/100) | (6/100) | (6/100) |

TABLE IB
Six, Nine & Twelve Month Continuous
Abstinence Rates By NicVAX ® Antibody Response

| | 6-Month | 12-Month | 12-Month |
|---|---|---|---|
| | (Wk 19-26) | (Wk 19-52) | 44-Week |
| NicVAX | 25% (n = 15/61) | 20% (n = 12/61) | 18% (n = 11/61) |
| High Antibody | p = 0.02 | p = 0.04 | p = 0.01 |
| | OR = 2.69 (1.14-6.37) | OR = 2.64 (1.03-6.79) | OR = 3.84 (1.32-11.20) |
| NicVAX | 9% (n = 13/140) | 7% (n = 10/140) | 7% (n = 10/140) |
| Low Antibody | p = 0.46 | p = 0.43 | p = 0.67 |
| | OR = 0.73 (0.31-1.71) | OR = 0.68 (0.26-1.76) | OR = 1.26 (0.43-3.65) |

### IA. Anti-Nicotine Antibody Compositions

Anti-nicotine antibody compositions also have been disclosed in the art. In the context of the present invention, anti-nicotine antibody compositions can be used as an alternative to or in addition to nicotine immunogenic compositions. For example, anti-nicotine antibodies can be administered directly to a subject to provide a serum antibody level, or to supplement the serum antibody level achieved in response to a nicotine immunogenic composition. The direct administration of anti-nicotine antibodies (also referred to as “passive immunization”) may permit a threshold serum antibody level to be reached more quickly than would be achieved by the administration of only a nicotine immunogenic composition. Additionally or alternatively, the direct administration of anti-nicotine antibodies may achieve higher serum antibody levels in a subject than could be achieved with only a nicotine immunogenic composition, such as if a nicotine immunogenic composition does not induce anti-nicotine antibodies in the subject, or produces a lower level of antibodies than desired.

The primary mechanism underlying the efficacy of both nicotine vaccines and passive immunization therapies with anti-nicotine antibodies is believed to be the binding of the anti-nicotine antibodies to nicotine, which sequesters the nicotine in the blood and prevents it from reaching the brain in high enough quantities to induce the release of dopamine. Anti-nicotine antibodies can be produced by the subject natively in response to a nicotine immunogenic composition (such as in response to active immunization with a nicotine immunogenic composition) or can be administered directly to the subject. Experiments reported in Pentel et al., Pharm. Biochem. & Behav. 65: 191-98 (2000), show that administered antibodies perform identically to induced antibodies with respect to capturing nicotine in the blood and reducing nicotine levels in the brain, when evaluated in both a pharmacological model and a functional model. In particular, purified antibodies from immunized rabbits administered to rats were shown to significantly increase the level of nicotine in the blood as compared to rats receiving rabbit immunoglobulin (IgG) from non-immunized rabbits. Further, the amount of nicotine reaching the brain was reduced significantly (64%) in animals receiving immune, anti-nicotine antibody-containing IgG, as compared to those receiving non-immune IgG. This effect was also shown to be nicotine-specific, IgG concentration-dependant, with higher concentrations of anti-nicotine antibodies sequestering more nicotine in the blood and reducing more brain content of nicotine.

1. Preparation of Anti-Nicotine Antibodies

As used herein, the term “anti-nicotine antibodies” means antibodies, including monoclonal and polyclonal antibodies, single chain antibodies, recombinant antibodies, and the like, that specifically bind nicotine, including IVIG preparations. Antibody fragments (e.g., Fab fragments) that specifically bind nicotine also can be used. Protocols for producing antibodies such as monoclonal antibodies are well known in the art and are described, for example, in Ausubel, et al. (eds.), Molecular Cloning: A Laboratory Manual, Cold Spring Harbor Laboratory, (Cold Spring Harbor, N.Y.)., Chapter 11; in METHODS OF HYBRIDOMA FORMATION 257-271, Bartal & Hirshaut (eds.), Humana Press, Clifton, N.J. (1988); in Vitetta et al., Immunol. Rev. 62:159-83 (1982); and in Raso, Immunol. Rev. 62:93-117 (1982). Protocols for producing anti-nicotine antibodies in particular are described for example, in U.S. Pat. No. 6,518,031, the entire contents of which are incorporated herein by reference in their entirety.

Polyclonal antibodies can be prepared as described above with reference to the nicotine immunogenic composition. For example, a nicotine-carrier conjugate, optionally in conjunction with an adjuvant, is diluted in a physiologically-tolerable diluent such as saline, to form an aqueous composition. An immunostimulatory amount of inoculum, with or without adjuvant, is administered to a mammal (e.g., a human) and the inoculated mammal is then maintained for a time period sufficient to produce anti-nicotine antibodies. Boosting doses of the nicotine-carrier conjugate composition may further enhance this process. Antibodies can be obtained by bleeding the animals and recovering serum or plasma for further processing. Antibodies can be prepared in humans or in a variety of commonly used animals, e.g., goats, primates, donkeys, swine, rabbits, horses, hens, guinea pigs, rats, and mice, after appropriate selection, fractionation and purification.

Antibodies can be harvested and isolated to the extent desired by well known techniques, such as by alcohol fractionation and column chromatography, or by immunoaffinity chromatography; that is, by binding antigen to a chromatographic column packing like Sephadex™, passing the antiserum through the column, thereby retaining specific antibodies and separating out other immunoglobulins (IgGs) and contaminants, and then recovering purified antibodies by elution with a chaotropic agent, optionally followed by steps to further purify the antibodies. This procedure may be followed when isolating the desired antibodies from the sera or plasma of humans that have developed an antibody titer against the antigen in question (e.g., a nicotine hapten), thus assuring the retention of antibodies that are capable of binding to nicotine.

A monoclonal antibody composition contains, within detectable limits, only one species of antibody that specifically binds to the antigen. Suitable monoclonal antibodies can be prepared—using conventional techniques, such as hybridoma technology or phage display technology. For example, to form hybridomas from which a monoclonal antibody composition is produced, a myeloma or other self-perpetuating cell line is fused with lymphocytes obtained from peripheral blood, lymph nodes or the spleen of a mammal hyperimmunized with the antigen. The myeloma cell line often is from the same species as the lymphocytes. Splenocytes are typically fused with myeloma cells using polyethylene glycol 1500. Fused hybrids are selected by their sensitivity to HAT. Hybridomas secreting the antibody molecules of this invention can be identified using an ELISA. Balb/C mouse spleen, human peripheral blood, lymph nodes or splenocytes are typically used in preparing murine or human hybridomas. Suitable mouse myelomas include the hypoxanthine-aminopterin-thymidine-sensitive (HAT) cell lines. One exemplary fusion partner for human monoclonal antibody production is SHM-D33, a heteromyeloma available from ATCC, Manassas, Va. under the designation CRL 1668. Fully human monoclonal antibodies or humanized monoclonal antibodies can be produced using these techniques.

A monoclonal antibody composition useful in accordance with the present invention can be produced by initiating a monoclonal hybridoma culture comprising a nutrient medium—containing a hybridoma that secretes anti-nicotine antibodies. The culture is maintained under conditions and for a time period sufficient for the hybridoma to secrete the antibody molecules into the medium. The antibody-containing medium is then collected, and the antibodies can be isolated further by well known techniques.

Alternatively, monoclonal antibodies can be cloned from hybridoma cells, phage display techniques or other known techniques, and inserted into an appropriate expression cell line that can express and produce high amounts of antibodies. Exemplary cell lines include Chinese Hamster Ovary cell line (CHO), insect cells, or other cell lines.

Other methods of preparing monoclonal antibody compositions are also contemplated, such as interspecies fusions. Those skilled in the art will appreciate that it is primarily the antigen specificity of the antibodies that affects their suitability for use in the context of the present invention. For example, human lymphocytes obtained from individuals treated with a nicotine immunogenic composition, can be fused with a human myeloma cell line to produce hybridomas which can be screened for the production of antibodies that specifically bind nicotine. Thus, a human treated with the nicotine immunogenic compositions described herein can serve as a source for either monoclonal or polyclonal antibodies to be used in an antibody composition as described herein.

As noted above, antibody compositions useful in accordance with the present invention may include whole antibodies, antibody fragments, and/or antibody subfragments. Antibodies can be whole immunoglobulin of any class, e.g., IgG, IgM, IgA, IgD, IgE, chimeric antibodies or hybrid antibodies with dual or multiple antigen or epitope specificities. Fragments can be F(ab′)₂, Fab′, Fab and the like, including hybrid fragments. Other immunoglobulins or natural, synthetic or genetically engineered proteins that act like an antibody by specifically binding to nicotine also can be used. In particular, Fab molecules can be expressed and assembled in a genetically transformed host like E. coli. A lambda vector system is available thus to express a population of Fab′s with a potential diversity equal to or exceeding that of subject generating the predecessor antibody. See Huse, W. D. et al., Science 246: 1275-81 (1989).

Antibody compositions can be prepared by formulating antibodies into compositions suitable for administration, such as by combining with a pharmaceutically acceptable excipient that may include other optional agents, such as discussed above with reference to nicotine immunogenic compositions.

Anti-nicotine antibodies can be modified to exhibit or improve any desired property, for example, to increase stability or in vivo half-life, or to make isolation/purification more efficient or effective, by methods that are well known in the art. One non-limiting exemplary modification includes PEGylation, e.g., conjugating the antibody to a polyethylene glycol moiety to increase in vivo circulation/half-life,

Anti-nicotine antibodies can be incorporated into a pharmaceutical composition for administration. Typically, such a composition will comprise the antibodies in an aqueous, saline solution at a pharmaceutically acceptable pH. However, it is possible to use an injectable suspension of the antibody. In addition to the usual pharmaceutically acceptable excipients, the composition may contain optional components to ensure purity, enhance bioavailability and/or increase penetration, such as described above for the nicotine immunogenic composition.

In specific embodiments, the anti-nicotine antibody composition is a pharmaceutical composition comprising anti-nicotine antibodies that is sterile and sufficiently stable to withstand storage, distribution, and use, and optionally includes additional components to protect the composition from infestation with, and growth of, microorganisms. Methods of preparing such compositions are well known to the skilled artisan and include, but are not limited to, vacuum drying, freeze-drying, and spin drying. These techniques yield a powder of the active ingredient along with any additional excipient incorporated into the pre-mix, which then can be formulated for administration by the desired route prior to use.

2. Use of Anti-Nicotine Antibodies

As noted above, in the context of the present invention, anti-nicotine antibody compositions can be used as an alternative to or in addition to nicotine immunogenic compositions, with or without the use of nicotine receptor agonists and/or antagonists. For example, anti-nicotine antibodies can be administered to a subject to provide a serum antibody level, or to supplement the serum antibody level achieved in response to a nicotine immunogenic composition. Thus, a threshold level of anti-nicotine antibodies can be achieved by administration of a nicotine immunogenic composition, by administration of anti-nicotine antibodies, or by administration of both a nicotine immunogenic composition and anti-nicotine antibodies (concurrently or sequentially). Since the administration of anti-nicotine antibodies results in a nearly immediate increase of serum antibody levels, the relative timing of administration of anti-nicotine antibodies and a nicotine immunogenic composition and/or nicotine receptor agonist and/or antagonist is flexible and can be adapted to suit the particular subject and method.

For example, in some embodiments, a subject is administered one or more doses of a nicotine immunogenic composition, and then the subject's serum anti-nicotine antibody levels are measured; if the level is below a threshold level, anti-nicotine antibodies can be administered to the subject, such as, for example, to achieve the threshold level. This methodology can be carried out as an independent method, or can be overlayed with a method that further comprises the administration of a nicotine receptor agonist and/or antagonist. Methods comprising administering anti-nicotine antibodies, measuring serum antibody levels and administering additional anti-nicotine antibodies if a threshold level has not been reached also can be carried out without the use of a nicotine immunogenic composition. For example, serum antibody levels (such as may be induced by a nicotine immunogenic composition and/or achieved by administration of anti-nicotine antibodies) could be measured and, if necessary, brought to a threshold level by the administration of anti-nicotine antibodies. Such methods may be carried out with or without the use of a nicotine receptor agonist and/or nicotine receptor antagonist. In accordance with some embodiments, serum anti-nicotine antibody levels are brought to a threshold level before the course of treatment with the nicotine receptor agonist and/or antagonist is commenced; during the course of treatment with the nicotine receptor agonist and/or antagonist; near the end of the course of treatment with the nicotine receptor agonist and/or antagonist, or after the course of treatment with the nicotine receptor agonist and/or antagonist, including before or after the target quit date, before or after smoking cessation or abstinence is achieved and/or before or after relapse, such as to achieve or maintain a threshold serum antibody level to treat nicotine addiction, promote smoking cessation, extend the duration of smoking abstinence in a subject who has quit smoking, increase the likelihood of long-term abstinence, prevent relapse (e.g., reduce the likelihood of a relapse) of nicotine consumption following a period of nicotine abstinence, and/or rescue a subject from relapse (including relapse in a subject who initially achieved abstinence while being treated in accordance with one of the methods described herein). In specific embodiments, a subject's anti-nicotine antibody levels are measured and, if the level is below a threshold level, anti-nicotine antibodies are administered to the subject. This process can be repeated until the threshold antibody level is achieved.

In some embodiments, a composition comprising anti-nicotine antibodies is administered in conjunction with a nicotine immunogenic composition, such as before, during or after administration of one or more doses of a nicotine immunogenic composition. For example, in some embodiments, one or more doses of an antibody composition is administered about 1 month before, about 1 week before, about 3 days before, or about one day before one or more doses of a nicotine immunogenic composition. In other embodiments, one or more doses of an antibody composition is administered during a course of treatment with a nicotine immunogenic composition. In further embodiments, one or more doses of an antibody composition is administered about 1 month after, about 2 weeks after, about 1 week after, about 3 days after or about 1 day after one or more doses of a nicotine immunogenic composition. In other embodiments, an antibody composition is administered after one, two, three or more doses of a nicotine immunogenic composition already have been administered, such as if the nicotine immunogenic composition was not effective to induce a threshold level of serum anti-nicotine antibodies, or if a shorter course of nicotine immunogenic composition is desired. For example, any time a booster dose of nicotine immunogenic composition might be administered, an anti-nicotine antibody compositions could be administered instead of or in addition to the nicotine immunogenic composition. As discussed above, a composition comprising anti-nicotine antibodies can be administered in conjunction with a nicotine immunogenic composition at any time relative to administration of the nicotine immunogenic composition, such as to achieve a threshold antibody level at any given time.

In some embodiments, a composition comprising anti-nicotine antibodies is administered independently of a nicotine immunogenic composition, before, during or after a course of treatment with a nicotine receptor agonist and/or antagonist. For example, in some embodiments, one or more doses of an anti-nicotine antibody composition is administered about 1 month before, about 1 week before, about 3 days before, or about one day before a course of treatment with a nicotine receptor agonist and/or antagonist. In other embodiments, one or more doses of an anti-nicotine antibody composition is administered during a course of treatment with a nicotine receptor agonist or antagonist. In further embodiments, one or more doses of an anti-nicotine antibody composition is administered about 1 month after, about 2 weeks after, about 1 week after, about 3 days after or about 1 day after a course of treatment with a nicotine receptor agonist or antagonist.

In some embodiments, a composition comprising anti-nicotine antibodies is administered before, during or after a course of treatment with a nicotine receptor agonist or antagonist, either without or further in conjunction with a nicotine immunogenic composition (and at any time relative to administration of a nicotine immunogenic composition as discussed above). For example, a composition comprising anti-nicotine antibodies can be administered after administration of one or more doses of a nicotine immunogenic composition (e.g., after two or three doses) and before or during or after a course of treatment with a nicotine receptor agonist or antagonist. For example, in some embodiments, an antibody composition is administered simultaneously with a nicotine receptor agonist and/or antagonist. In other embodiments, an antibody composition is administered near the end or at the end of a course of treatment with a nicotine receptor agonist and/or antagonist. In some embodiments, administration of the antibody composition simultaneous with a nicotine receptor agonist and/or antagonist may increase the chances of a successful quit (e.g., promote smoking cessation), while administration of an antibody composition near the end of a course of treatment with a nicotine receptor agonist and/or antagonist may enhance relapse prevention for all subjects, and may be especially beneficial for subjects who have already quit nicotine consumption by the end of the course of treatment with a nicotine receptor agonist and/or antagonist, such as by preventing relapse and/or extending the duration of smoking abstinence. Administration of an antibody composition near the end or after a course of treatment with a nicotine receptor agonist and/or antagonist also may enhance rescue for subjects who have not quit nicotine consumption by the end of the course of treatment with a nicotine receptor agonist and/or antagonist.

The use of an anti-nicotine antibody composition in conjunction with a nicotine immunogenic composition (in accordance with some embodiments) may offer benefits, for example, by alleviating effects of nicotine while the subject's immune system responds to the nicotine immunogenic compound. As noted above, the direct administration of anti-nicotine antibodies may permit a threshold serum antibody level to be reached more quickly than would be achieved by the administration of only a nicotine immunogenic composition. Additionally, as discussed above, the administration of anti-nicotine antibodies can be tailored to achieve a threshold serum antibody level quickly, or can be tailored to mimic the gradual increase that would be achieved with a nicotine immunogenic composition, whichever may be desired for a specific subject or target patient population. Additionally or alternatively, the direct administration of anti-nicotine antibodies may achieve higher serum antibody levels in a subject than could be achieved with only a nicotine immunogenic composition, such as if a nicotine immunogenic composition does not induce anti-nicotine antibodies in the subject, or produces a lower level of antibodies than desired.

Anti-nicotine antibodies can be administered by any suitable route, e.g., intranasal, intradermal, subcutaneous, intramuscular or intravenous. The amount of antibody administered will depend on a variety of factors, such as patient weight, age, overall health, anti-nicotine antibody titer, and desired threshold antibody level. In some embodiments, suitable amounts include less than about 1 mg per kg, about 1 mg per kg, about 10 mg per kg, about 20 mg per kg, about 30 mg per kg, about 40 mg per kg, about 50 mg per kg, about 60 mg per kg, about 70 mg per kg, about 80 mg per kg, about 90 mg per kg, about 100 mg per kg, about 150 mg per kg, about 200 mg per kg, about 300 mg per kg, about 400 mg per kg, or about 500 mg per kg, of bodyweight of anti-nicotine antibodies are administered to a subject. Such amounts may be administered as a single dose or divided between multiple doses administered for example approximately 2, 4, 8, 12, 18 or 24 hours apart, or 2, 4, 6, 8, 10, 12, 14, 16, 18, 20, 22, 24, 26, 28, 30 days apart, or once per week, once every two weeks, once every three weeks, once every four weeks, once every five weeks, once every six weeks, etc., In other embodiments, doses are administered at irregular intervals. Antibody doses can also be administered on a schedule similar to that of the immunogenic composition, and can be adjusted to achieve a threshold antibody level.

### II. NICOTINE RECEPTOR AGONISTS

In general, nicotine receptor agonists act by binding to the nicotine receptor, preventing nicotine molecules from effectively occupying the same receptor, thereby dampening or blocking a nicotine-medicated response in the brain. As used herein, the term “nicotine receptor agonist” does not include nicotine.

One non-limiting example of a nicotine receptor agonist is varenicline, IUPAC name 7,8,9,10-tetrahydro-6,10-methano-6H-pyrazino[2,3-h][3]benzazpine, (2R,3R)-2,3-dihydroxybutanedioate(1:1), sold under the trade name CHANTIX® (also known as CHAMPIX outside the U.S.) by Pffizer. Varenicline is a partial agonist selective for α4β2 nicotinic acetylcholine receptor subtypes. Varenicline, as the tartrate salt, is a powder which is a off-white to slightly yellow solid which is highly soluble in water. Varenicline tartrate has a molecular weight of 361.35 Daltons and has molecular formula C₁₃H₁₃N₃—C₄H₆O₆. The chemical structure of varenicline is shown below.

Varenicline has been shown to bind the α4β2 nicotinic receptors and act as a low level agonist (i.e., stimulating receptor-mediated activity but at a significantly lower level than nicotine) while simultaneously preventing nicotine binding the α4β2 receptor. Because varenicline blocks the ability of nicotine to activate the α4β2 receptors and thus to stimulate the central nervous mesolimbic dopamine system, the reinforcement and reward experienced by smoking is diminished. Varenicline also may act on other receptors in the brain, which binding also may aid in smoking cessation.

CHANTIX® tablets currently are supplied for oral administration in two strengths: a 0.5 mg capsule and a 1.0 mg capsule. The currently recommended dose of CHANTIX® currently is 1 mg twice daily following a 1-week titration as follows: Days 1-3: 0.5 mg once daily; Days 4-7: 0.5 mg twice daily; Days 8-End of treatment: 1 mg twice daily (bid). A course of CHANTIX® treatment generally lasts for 12 weeks.

Side effects of CHANTIX® include, inter alia, neuropsychiatric events (including but not limited to depression, mania, psychosis, hallucinations, paranoia delusions, suicidal ideation, suicide attempt and complete suicide), nausea, somnolence, dizziness, loss of consciousness, difficulty concentrating, hot flushes, hypertension, angioderma and skin reactions.

When used alone, e.g., not in accordance with the present invention, subjects are generally instructed to quit smoking one week after starting CHANTIX®, and subjects who have successfully stopped smoking at the end of 12 weeks, may be advised to follow an additional 12 week course of treatment with CHANTIX® to increase the likelihood of long term abstinence.

Two independent studies compared the efficacy of 12 week courses of CHANTIX®, bupropion and placebo, and assessed carbon-monoxide confirmed continuous abstinence rates during-weeks 9-12.

TABLE II
Continuous Abstinence Rate (Wks 9-12) (95% Confidence Interval)

| | Varenicline | Bupropion | |
|---|---|---|---|
| Study | (1.0 mg bid) | (150 mg SR bid) | Placebo |
| Study A | 21% | 16% | 8% |
| | (17%, 26%) | (12%, 20%) | (5%, 11%) |
| Study B | 22% | 14% | 10% |
| | (17%, 26%) | (11%, 18%) | (7%, 13%) |

### III. NICOTINE RECEPTOR ANTAGONIST

In general, nicotine receptor antagonists preventing nicotine molecules from effectively occupying nicotine receptors, thereby dampening or blocking a nicotine-medicated response in the brain.

One non-limiting example of a nicotine receptor antagonist is bupropion hydrochloride, sold under the trade name ZYBAN® (Glaxo-Wellcome). Bupropion hydrochloride is also sold under the name WELLBUTRIN® which is prescribed as an antidepressant. Bupropion hydrochloride has molecular formula C₁₃H₁₈ClNO—HCl, the powder is white, crystalline, and highly soluble in water. The chemical structure of bupropion hydrochloride is shown below.

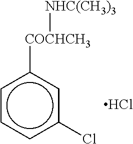

ZYBAN® tablets currently are supplied for oral administration as 150 mg sustained release tablets.

The currently recommended and maximum dose of ZYBAN® is 300 mg/day, given as 150 mg twice daily (bid). The current dosing regimen includes beginning dosing at 150 mg/day every day for the first 3 days, followed by a dose increase for most subjects to the recommended usual dose of 300 mg/day, with subjects advised to have an interval of at least 8 hours between successive doses, and doses above 300 mg/day not recommended. A course of ZYBAN® treatment generally lasts for 7 to 12 weeks. Dose tapering of ZYBAN® is not required when discontinuing treatment.

Side effects of ZYBAN® include, inter alia, depression, suicidal ideation, suicide attempt, complete suicide, mania, psychosis, hallucinations, paranoia, delusions, seizures, fever, itching, rash, nausea, stomach upset, vomiting, difficulty concentrating, headache, dizziness, insomnia, agitation, hypertension, tremor and sweating.

When used alone, e.g., not in accordance with the present invention, subjects are generally advised to set a “target quit date” within the first 2 weeks of treatment with ZYBAN®, generally in the second week, because approximately 1 week of treatment is required to achieve steady-state blood levels of bupropion. When used alone, subjects who have not made significant progress towards abstinence by the seventh week of therapy with ZYBAN® are unlikely to succeed, and treatment is generally discontinued. Conversely, a subject who successfully quits after 7 to 12 weeks of treatment may be advised to continue therapy with ZYBAN®.

Bupropion acts as a broad-spectrum non-competitive nicotine receptor antagonist, blocking nicotine activation of α3β2, α4β2 and α7 nicotinic acetylcholine receptors (nAChRs) with some degree of selectivity. Bupropion was found to be 50 and 12 times more effective in blocking α3β2 and α4β2 than α7. Bupropion also acts on non-nicotine receptors and exhibits anti-anxiety activity, and is a relatively weak inhibitor of the neuronal uptake of norepinephrine and dopamine, and does not inhibit monoamine oxidase or the re-uptake of serotonin.

Studies have demonstrated that subjects taking bupropion double the chances of quitting smoking compared with placebo. Bupropion has also been shown to lower nicotine craving and nicotine withdrawal symptoms. Table 2 below shows the results of a clinical trail with ZYBAN®. Sample size for each group was N=151 for placebo, N=153 for 100 mg/day and 150 mg/day, and N=156 for 300 mg/day.

TABLE III
ZYBAN quit data

| Abstinence from | Treatment groups | | | |
|---|---|---|---|---|
| week 4 through | | ZYBAN | ZYBAN | ZYBAN |
| specified week | Placebo | 100 mg/day | 150 mg/day | 300 mg/day |
| Week 7 | 17% | 22% | 27% | 36% |
| (4-week quit) | | | | |
| Week 12 | 14% | 20% | 20% | 25% |
| Week 26 | 11% | 16% | 18% | 19% |

### IV. OTHER SMOKING-CESSATION AGENTS

In some embodiments, one or more other smoking-cessation agents are used as an alternative to, or in addition to, a nicotine receptor agonist and/or antagonist. Examples of such other smoking-cessation agents include, but are not limited to, one or more of a nicotinic cholinergic antagonist (such as mecylamine), monoamine oxidase inhibitors, glycine antagonists, opiate antagonists and agonists, dopamine D3 antagonists, nicotinic ligands, dopamine uptake inhibitors, cannabinoid receptor 1 antagonists, inhibitors of enzymes involved in nicotine and/or cotinine metabolism, including cytochrome P450 enzymes (e.g., cytochrome p450 2A6-CYP2A6), aldehyde oxidase, flavin-containing monooxygenase 3, amine N-methyltransferase, and UDP-glucuronosyltransferases.

### V. COMBINATION THERAPY

As discussed above, the present methods relate to administering a combination of a nicotine immunogenic composition (e.g., vaccine) and/or anti-nicotine antibodies as described above and/or a nicotine receptor antagonist and/or a nicotine receptor agonist (or other anti-nicotine agent) as described above to treat nicotine addiction and nicotine-addiction related disorders, promote smoking cessation, extend the duration of smoking abstinence in a subject who has quit smoking, increase the likelihood of long-term abstinence, prevent relapse (e.g., reduce the likelihood of a relapse) of nicotine consumption following a period of nicotine abstinence, and/or rescue a subject from relapse (including relapse in a subject who initially achieved abstinence while being treated in accordance with the method). In accordance with some embodiments, the nicotine immunogenic composition and/or anti-nicotine antibody composition is administered in a course that has been determined to be effective when used independently. In accordance with some embodiments, the nicotine agonist or antagonist is administered in a course that has been determined to be effective when used independently. In accordance with other embodiments, the nicotine agonist or antagonist is administered at a dosage lower than that determined to be effective when used independently. As discussed above, in some embodiments, the courses are administered such that they overlap or such that the effects of the different therapies overlap, as described in more detail above and below.

Although any of the nicotine receptor agonists and/or antagonists or other smoking-cessation agents described above may be used in accordance with specific embodiments of the methods described herein, in accordance with other specific embodiments, the methods described herein do not include the administration of one or more of the disclosed agents. For example, in some specific embodiments, the methods do not comprise the administration of one or more of varenicline; an anti-depressant, such as bupropion (ZYBAN®, WELLBUTRIN®), fluoxetine (Prozac, nortriptyline, doxepin, desipramine, clomipramine, imipramine, amitriptyline, trimipramine, fluvoxamine, proxetine, sertraline, phenelzine, tranylcypromine, amoxapine, maprotiline, trazodone, venlafaxine, or mirtrazapine; (5aS,8S,10aR)-5a,6,9,10-tetrahydro, 7H,11H-8,10a-methanopyrido[2′,3′:5,6]py-rano-[2,3-d]azepine (SSR591813); a nicotine receptor antagonist, such as mecamylamine, amantadine, pempidine, dihydro-beta-erthyroidine, hexamethonium, erysodine, chlorisondamine, trimethaphan camsylate, tubocurarine chloride, and d-tubocurarine; or a monoamine oxidase inhibitor.

In some embodiments, the course of nicotine immunogenic composition comprises a single dose or multiple doses over a pre-determined period of time. In some embodiments, such as where a single dose is used, the nicotine immunogenic composition may be formulated as an extended release composition. In some embodiments, the subject is administered a first dose of a nicotine immunogenic composition (nicotine vaccine), followed by one or more “booster” doses. In some embodiments, a course of treatment may include a single dose, or one, two, three, four, five or six booster doses following an initial dose of the nicotine vaccine. In some embodiments, four booster doses are provided after the initial vaccination. In some embodiments, five booster doses are provided after the initial vaccination. A course of treatment ends when the last booster dose is provided. A second or subsequent course may commence after a first or other previous course has ended, such as six months or longer after the last dose of a previous course. Additionally or alternatively, one or more further boosters may be provided six months or longer after a given course of treatment, such as six months, 1 year, 18 months, 2 years, 3 years, 4, years, 5 years or longer after the last dose.

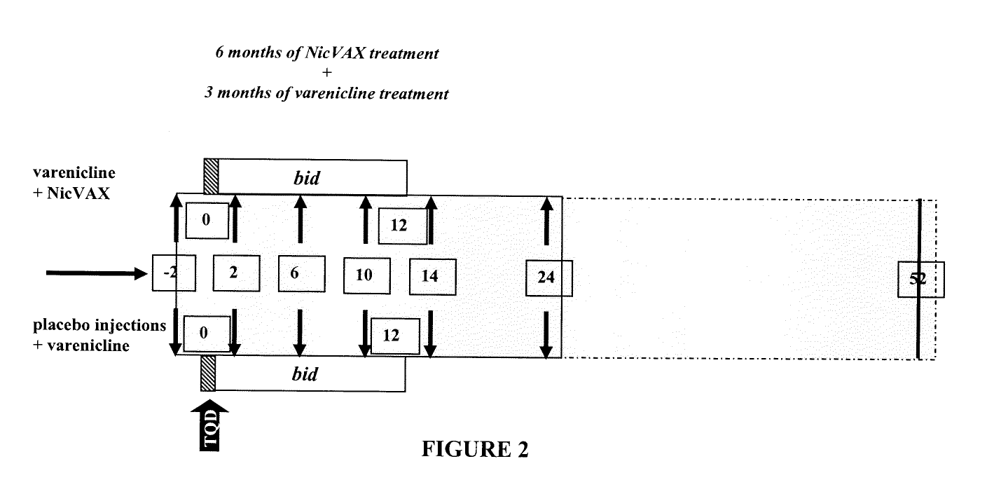

The course of administration may reflect a single administration (single dose) or may extend over a predetermined period of time, such as about 4 weeks, about 6 weeks, about 8 weeks, about 10 weeks, about 12 weeks, about 14 weeks, about 16 weeks, about 18 weeks, about 20 weeks, about 22 weeks, about 24 weeks, about 26 weeks (e.g., about 6 months), about 28 weeks, about 30 weeks, or longer. In one embodiment, the course extends over a predetermined period of time of about 24 to 26 weeks, including 24 to 26 weeks, such as 24, 25 or 26 weeks (e.g., about 6 months, such as 6 months). In some embodiments, the dosages are administered at regular intervals during a course of treatment. For example, in some embodiments, boosters are administered once per week, once every two weeks, once every three weeks, once every four weeks, once every five weeks, once every six weeks, etc., during a course of treatment. In other embodiments, boosters are administered at irregular intervals during a course of treatment. For example, in one embodiment, boosters are administered (relative to the first dose at time 0) at 4 weeks, 8 weeks, 16 weeks, and 26 weeks. In another embodiment, the boosters are administered (relative to the first dose at time 0) at 6 weeks, 12 weeks and 16 weeks, and optionally at 26 weeks. In another embodiment, the boosters are administered (relative to the first dose at time 0) at 4 weeks, 8 weeks, 12 weeks, 16 weeks and 26 weeks, as illustrated in FIG. 2. In some embodiments, the timing of a second or subsequent dosage is selected with reference to the subject's level of anti-nicotine antibodies, as described in U.S. patent application Ser. No. 12/481,420, filed Jun. 9, 2009, the entire contents of which are incorporated herein by reference in their entirety, and international patent application PCT/US09/47679, filed Jun. 9, 2009, the entire contents of which are incorporated herein by reference in their entirety.

The booster doses can include the same or a different immunogenic composition compared to the first dose (e.g., such as by comprising a different antigenic component or different formulation or being administered by a different route of administration), and can include the use of one or more different immunogenic compositions or multivalent compositions. In some embodiments, the nicotine immunogenic composition NicVAX® is administered throughout an entire course of treatment.

The dosage of the nicotine immunogenic composition administered in the various doses may be the same as, greater than, or lower than, the dosage of any nicotine immunogenic composition previously administered to the subject. The particular dosage may vary with the nicotine immunogenic composition being used, as discussed above. In some embodiments, the nicotine immunogenic composition is administered in dosages that have been determined to be effective when used independently. In some embodiments, a dose provides at least about 5 μg nicotine hapten, such as 5 μg nicotine hapten. In some embodiments, a dose provides at least about 10 μg nicotine hapten, such as 10 μg nicotine hapten. In some embodiments, a dose provides about 16 μg nicotine hapten+/−5 μg nicotine hapten, including about 9 to 21 μg nicotine hapten, and 9 to 21 μg nicotine hapten, such as 9, 10, 12, 14, 16, 18, 20, or 21 μg nicotine hapten. As discussed above, the potency of a nicotine immunogenic composition may depend on the specific hapten, the specific carrier, whether an adjuvant is present and, if so, its potency, and the route of administration. Thus, the dosage guidelines provided herein are exemplary only, and suitable doses of a given nicotine immunogenic composition can be determined by the skilled practitioner. In some embodiments, NicVAX® is administered for each immunization in a given course of treatment at a dose of about 400 μg, based on the amount of carrier protein administered. In some embodiments, NicVAX® is administered for each immunization in a given course of treatment at a dose of about 200 μg, based on the amount of carrier protein administered. In some embodiments, NicVAX® is administered for one or more immunizations, including all immunizations, in a given course of treatment at dose of greater than about 400 μg or less than about 200 μg, based on the amount of carrier protein administered.

As discussed above, an anti-nicotine antibody composition can be used as an alternative to or in addition to a nicotine immunogenic composition, in order to achieve a level of anti-nicotine antibodies in the subject. Thus, one or more doses of an anti-nicotine antibody composition can be administered instead of a course of nicotine immunogenic composition. Alternatively, one or more doses of an anti-nicotine antibody composition can be administered in addition to a course of nicotine immunogenic composition. In yet another alternative, one or more doses of an anti-nicotine antibody composition can be administered instead of one or more doses of nicotine immunogenic composition, with one or more doses of nicotine immunogenic composition also being administered.

In general, the more doses of a nicotine vaccine that a subject receives within a single course of treatment (e.g., within six months), and/or the higher the dose of anti-nicotine antibodies, the higher the individual's antibody levels will become, and hence the greater the likelihood of successful treatment of nicotine addiction, successful smoking cessation, successful extension of the duration of smoking abstinence, reduced likelihood of relapse, increased likelihood of rescue from relapse, and increased likelihood of successful achievement of long-term abstinence.

In accordance with some embodiments, the nicotine receptor antagonist and/or agonist is administered as a course of treatment as discussed above, such as a course that has been determined to be effective when used independently. For example, in some embodiments varenicline and/or bupropion are administered daily (typically in one or two divided doses) for about 12 weeks.

As discussed above, in accordance with some embodiments of the methods described herein, the course of nicotine immunogenic composition and/or anti-nicotine antibodies overlaps with the course of nicotine receptor agonist and/or antagonist. In other embodiments, the effect of the nicotine immunogenic composition and/or anti-nicotine antibody overlaps with the course of the nicotine receptor antagonist and/or agonist. In general, the relative timing of the course of the nicotine immunogenic composition and/or anti-nicotine antibodies and the course of the nicotine receptor agonist and/or antagonist is such that a first threshold level of anti-nicotine antibodies is achieved in the subject (e.g., induced by the nicotine immunogenic composition and/or by administration of anti-nicotine antibodies) by the time that the course of nicotine receptor agonist and/or antagonist is completed and/or by the time that the efficacy of the nicotine receptor agonist or antagonist is diminishing or has diminished. As noted above, in some embodiments, exemplary first threshold serum antibody levels include at least about 6 μg/ml, at least about 10 μg/ml, at least about 12 μg/ml, at least about 15 μg/ml, at least about 20 μg/ml, at least about 25 μg/ml, at least about 30 μg/ml, at least about 35 μg/ml, at least about 40 μg/ml, at least about 45 μg/ml, or, at least about 50 μg/ml, such as at least 6 μg/ml, at least 10 μg/ml, at least 12 μg/ml, at least 15 μg/ml, at least 20 μg/ml, at least 25 μg/ml, at least 30 μg/ml, at least 35 μg/ml, at least 40 μg/ml, at least 45 μg/ml, or at least 50 μg/ml. In other embodiments, the threshold serum anti-nicotine antibody level (in μg/ml) is from at least about 1.5 to at least about 2.0 times the number of cigarettes smoked per day by the subject, such as being 1.5 to 2.0 times the number of cigarettes smoked per day. In other embodiments, the first minimum threshold level is directly correlated with the number of doses of a nicotine immunogenic composition that the subject has received prior to the measuring of the level of anti-nicotine antibodies. For instance, the first specified threshold anti-nicotine antibody level can be selected from at least about 10 μg/ml for up to two prior doses (such as from at least 10 to at least 25 μg/ml), at least about 25 μg/ml for three prior doses (such as from at least 25 to at least 50 μg/ml), at least about 50 μg/ml for four prior doses (such as from at least 50 to at least 75 μg/ml), and at least about 60 μg/ml for five or more prior doses (such as from at least 60 to at least 75 or at least 100 μg/ml). The practitioner readily can determine corresponding secreted antibody levels using routine methodologies.

Thus, in accordance with the some embodiments of the methods described herein the relative timing of the course of the nicotine immunogenic composition and/or anti-nicotine antibodies and the course of the nicotine receptor agonist and/or antagonist is such that such a threshold level of anti-nicotine antibodies is achieved in the subject by the time that the course of nicotine receptor agonist or antagonist is completed and/or by the time that the efficacy of the nicotine receptor agonist or antagonist (in the context of, for example, smoking cessation) is diminishing or has diminished. For example, as discussed above, a course of nicotine receptor agonist or antagonist typically has a duration of about 12 weeks. With such a course of nicotine receptor agonist or antagonist, the relative timing of the course of the nicotine immunogenic composition and/or anti-nicotine antibodies and the course of the nicotine receptor agonist or antagonist may be selected such that a first threshold level of anti-nicotine antibodies is achieved in the subject by the 12th week of administration of the nicotine receptor agonist or antagonist (or earlier), such as by 6th through the 12th week, including by the 6th, 7th, 8th, 9th, 10th, 11th, or 12th week of administration of the nicotine receptor agonist or antagonist, or even earlier, such as by the 1st, 2nd, 3rd, 4th or 5th week of administration of the nicotine receptor agonist or antagonist. Of course, if the duration of the course of administration of the nicotine receptor agonist or antagonist is adjusted upwards or downwards, the timing of the achievement of the first threshold level of anti-nicotine antibodies may be adjusted accordingly. As discussed above, the efficacy of a nicotine receptor agonist or antagonist may diminish over its course of administration, sometimes despite ongoing administration, as has been reported for bupropion, which has been reported to exhibit diminished efficacy during about the 5th week through about the 7th week of administration. When the efficacy of a nicotine receptor agonist or antagonist diminishes over its course of administration, the relative timing of the course of the nicotine immunogenic composition and/or anti-nicotine antibodies and the course of the nicotine receptor agonist or antagonist may be selected such that a first threshold level of anti-nicotine antibodies is achieved in the subject by the time that the efficacy of the nicotine receptor agonist or antagonist (in the context of, for example, smoking cessation) is diminishing or has diminished, such as by about the 5th week through about the 7th week of administration of bupropion.

In some embodiments, the course of the nicotine immunogenic composition is started before the course of the nicotine receptor agonist or antagonist is started. In other embodiments, the course of the nicotine immunogenic composition is started substantially simultaneously with the course of the nicotine receptor agonist or antagonist. In some embodiments, the course of the nicotine immunogenic composition is started after the course of the nicotine receptor agonist or antagonist is started. For example, in one embodiment, the first dose of a course of a nicotine vaccine is administered two weeks or longer before the first dose of a nicotine receptor agonist and/or antagonist is administered. In other embodiments, the first dose of a course of a nicotine vaccine is administered substantially simultaneously with the first dose of a nicotine receptor agonist and/or antagonist. FIG. 2 illustrates an exemplary dosing regimen of a combination therapy in accordance with the invention. In accordance with the illustrated embodiment, a nicotine immunogenic composition is administered in a course of 6 dosages at weeks −2, 2, 6, 10, 14 and 24, relative to the course of administration of a nicotine agonist, with a nicotine agonist administered in a course of twice daily dosages for 12 weeks starting at week 0. As described above, the invention also includes methods where anti-nicotine antibodies are administered instead of a nicotine vaccine or in addition to a nicotine vaccine at any one or more of these time periods, or prior or subsequent to any one or more of these time periods.

In accordance with some embodiments of the methods described herein, anti-nicotine antibodies are administered alone or in combination with a nicotine immunogenic composition to achieve a first threshold level of anti-nicotine antibodies. Exemplary first threshold serum antibody levels include those discussed above, e.g., at least about 6 μg/ml, at least about 10 μg/ml, at least about 12 μg/ml, at least about 15 μg/ml, at least about 20 μg/ml, at least about 25 μl g/ml, at least about 30 μg/ml, at least about 35 μg/ml, at least about 40 μg/ml, at least about 45 μg/ml, or, at least about 50 μg/ml, such as at least 6 μg/ml, at least 10 μg/ml, at least 12 μg/ml, at least 15 μg/ml, at least 20 μg/ml, at least 25 μg/ml, at least 30 μg/ml, at least 35 μg/ml, at least 40 μg/ml, at least 45 μg/ml, or at least 50 μg/ml, or threshold serum anti-nicotine antibody levels that are (in μg/ml) from at least about 1.5 to at least about 2.0 times the number of cigarettes smoked per day by the subject, such as being 1.5 to 2.0 times the number of cigarettes smoked per day. In other embodiments, the first minimum threshold level is directly correlated with the number of doses of a nicotine immunogenic composition that the subject has received prior to the measuring of the level of anti-nicotine antibodies. For instance, the first specified threshold anti-nicotine antibody level can be selected from at least about 10 μg/ml for up to two prior doses (such as from at least 10 to at least 25 μg/ml), at least about 25 μg/ml for three prior doses (such as from at least 25 to at least 50 μg/ml), at least about 50 μg/ml for four prior doses (such as from at least 50 to at least 75 μg/ml), and at least about 60 μg/ml for five or more prior doses (such as from at least 60 to at least 75 or at least 100 μg/ml). The practitioner readily can determine corresponding secreted antibody levels using routine methodologies.

As noted previously, the administration of anti-nicotine antibodies has near immediate effect on serum antibody levels, and so the timing of administration of anti-nicotine antibodies can be controlled and selected to meet the needs of a particular subject in the context of a particular method. As discussed above, the administration of anti-nicotine antibodies can be tailored to achieve a threshold serum antibody level quickly, or can be tailored to mimic the gradual increase that would be achieved with a nicotine immunogenic composition, whichever may be desired for a specific subject or target patient population.

The methods described herein may further comprise selecting a target quit date, or counseling the subject on a suitable target quit date, and the kits described herein may include instructions on a suitable target quit date. In some embodiments, a target quit date is about 1-2 weeks after the first dose of nicotine receptor agonist or antagonist, such as one week, two weeks, or longer, after the first dose of nicotine receptor agonist or antagonist, up to about 12 weeks after the first dose of nicotine receptor agonist or antagonist, such as up to 12 weeks after the first dose of nicotine receptor agonist or antagonist. In some embodiments, a target quit date is about 1 week, 2 weeks, or longer, after the first dose of nicotine immunogenic composition and/or anti-nicotine antibodies, such as 1 week, 2 weeks, 3 weeks, 4 weeks, 5 weeks, 6 weeks, 7 weeks, 8 weeks, or longer, after the first dose of nicotine immunogenic composition and/or anti-nicotine antibodies, or any time before, after, or coincident with any subsequent (booster) dose of nicotine immunogenic composition and/or anti-nicotine antibodies. In some embodiments, a target quit date is about 1 week, 2 weeks, or longer, before or after a subsequent dose of nicotine immunogenic composition and/or anti-nicotine antibodies, such as 1 week, 2 weeks, 3 weeks, 4 weeks, 5 weeks, 6 weeks, 7 weeks, 8 weeks, or longer, before or after any subsequent (booster) dose of nicotine immunogenic composition and/or anti-nicotine antibodies, or any time before, after, or coinciding with any further subsequent (booster) dose of nicotine immunogenic composition and/or anti-nicotine antibodies. In some embodiments, a target quit date is determined with reference to the subject's level of anti-nicotine antibodies (induced via a nicotine immunogenic composition and/or achieved via administration of anti-nicotine antibodies), as described in U.S. patent application Ser. No. 12/481,420, filed Jun. 9, 2009, the entire contents of which are incorporated herein by reference in their entirety, and international patent application PCT/US09/47679, filed Jun. 9, 2009, the entire contents of which are incorporated herein by reference in their entirety, such as to coincide with achievement of a threshold level of serum anti-nicotine antibodies, as discussed herein. In accordance with the embodiment illustrated in FIG. 2, the target quit date is at about week 1, e.g., about one week after the course of nicotine agonist is commenced.

In some embodiments, a target quit date is timed relative to the administration of a dose of nicotine immunogenic composition and/or anti-nicotine antibodies, such as being about 1 week, 2 weeks, or longer, before or after a booster dose of nicotine immunogenic composition and/or anti-nicotine antibodies, such as 1 week, 2 weeks, 3 weeks, 4 weeks, 5 weeks, 6 weeks, 7 weeks, 8 weeks, or longer, before or after any subsequent (booster) dose of nicotine immunogenic composition and/or anti-nicotine antibodies, or any time before, after, or coinciding with any booster dose of nicotine immunogenic composition and/or anti-nicotine antibodies. In some embodiments, a target quit date is at least about 1 week after, such as 1 week after, the most recent dose of nicotine immunogenic composition and/or anti-nicotine antibodies. In some embodiments, a target quit date is at least about 1 week before, such as 1 week before, the next scheduled dose of nicotine immunogenic composition and/or anti-nicotine antibodies. In some embodiments, a target quit date is at least about one week after the most recent dose of nicotine immunogenic composition and/or anti-nicotine antibodies and at least about one week before the next scheduled dose of nicotine immunogenic composition and/or anti-nicotine antibodies, such as at least one week after the most recent dose of nicotine immunogenic composition and/or anti-nicotine antibodies and at least one week before the next scheduled dose of nicotine immunogenic composition and/or anti-nicotine antibodies. In other embodiments, a target quit date is about 2 weeks, 3 weeks, 4 weeks, 5 weeks, 6 weeks, 7 weeks, 8 weeks, or longer, after the most recent dose of nicotine immunogenic composition and/or anti-nicotine antibodies and/or about 2 weeks, 3 weeks, 4 weeks, 5 weeks, 6 weeks, 7 weeks, 8 weeks, or longer, before the next scheduled dose of nicotine immunogenic composition and/or anti-nicotine antibodies. For example, in embodiments where a nicotine immunogenic composition and/or anti-nicotine antibodies is administered at weeks 0, 4, 8, 12, 16 and 26, one or more target quit dates may be set at weeks 0, 2, 4, 6, 10, 14, 18, and 28, or at weeks 14, 18, and 28. As noted above, because the administration of anti-nicotine antibodies has near immediate effect on serum antibody levels, the target quit day can be set to coincide with the administration of anti-nicotine antibodies, or to be shortly thereafter as may be practical or convenient for the subject.

In some embodiments, multiple target quit dates are selected.

In specific embodiments, a subject's anti-nicotine antibody levels (such as serum antibody levels) are measured prior to or coincident with a target quit date and, if the level is below a threshold level, a nicotine immunogenic composition and/or anti-nicotine antibodies are administered to the subject. This process can be repeated until the threshold antibody level is achieved, at which point a target quit date can be set. In accordance with these embodiments, the subject will have achieved a threshold level of anti-nicotine antibodies prior to the target quit date.

In some embodiments, an initial target quit date is about 1-2 weeks after the first dose of nicotine receptor agonist or antagonist, such as one week or two weeks after the first dose of nicotine receptor agonist or antagonist. For subjects who do not quit smoking at the initial target quit date, or who relapse, a subsequent target quit date may be determined with reference to the first dose of nicotine immunogenic composition and/or anti-nicotine antibodies, or with reference to any subsequent dose of nicotine immunogenic composition and/or anti-nicotine antibodies, as described above, such as being about 1 week, 2 weeks, or longer, after the first dose, or before or after a subsequent dose, of nicotine immunogenic composition and/or anti-nicotine antibodies, such as 1 week, 2 weeks, 3 weeks, 4 weeks, 5 weeks, 6 weeks, 7 weeks, 8 weeks, or longer, after the first dose of nicotine immunogenic composition and/or anti-nicotine antibodies, or before or after any subsequent dose of nicotine immunogenic composition and/or anti-nicotine antibodies, or coinciding with any subsequent (booster) dose of nicotine immunogenic composition and/or anti-nicotine antibodies, as discussed above. In some embodiments a subsequent target quit date may be determined with reference to the subject's level of anti-nicotine antibodies, as described in U.S. patent application Ser. No. 12/481,420, filed Jun. 9, 2009, the entire contents of which are incorporated herein by reference in their entirety, and international patent application PCT/US09/47679, filed Jun. 9, 2009, the entire contents of which are incorporated herein by reference in their entirety, such as to coincide with achievement of a threshold level of serum anti-nicotine antibodies, as discussed herein.

Thus, in accordance with the methods described herein, subjects who do not make a successful quit attempt at the initial target quit date, or who relapse, may nevertheless be successfully treated without the need for a second course of nicotine receptor agonist or antagonist. Thus, in some embodiments, the methods described herein include only a single course of nicotine receptor agonist or antagonist treatment, such as a single 12 week course of administrations of nicotine receptor agonist or antagonist. The invention includes other embodiments, however, where multiple courses of nicotine receptor agonist or antagonist are administered, including multiple courses of the same nicotine receptor agonist or antagonist and/or courses with different nicotine receptor agonists and/or different antagonists. Each course of treatment may be the same or different than a previous course of treatment. Non-limiting examples of differences between courses of treatment include the duration of a course, the dosage provided with each administration within the course, the timing of administration, the composition administered, etc.

As discussed above, in accordance with the methods described herein, subjects may quit smoking earlier than subjects treated with a nicotine immunogenic composition alone, anti-nicotine antibodies alone, or nicotine receptor agonists and/or antagonists alone. Thus, a subject's likelihood of quitting smoking may increase as compared to the likelihood with treatment with only one component of the combination treatment (e.g., treatment with a nicotine immunogenic composition but not an anti-nicotine antibody or a nicotine receptor agonist or antagonist, or treatment with a nicotine receptor agonist or antagonist but not a nicotine immunogenic composition or anti-nicotine antibody, of treatment with an anti-nicotine antibody but not a nicotine immunogenic composition or a nicotine receptor agonist or antagonist). Moreover, the methods described herein may extend the duration of smoking abstinence in a subject who has quit smoking (including a subject who has quit smoking while being treated in accordance with the method), may increase the likelihood of long-term abstinence, may prevent (e.g., reduce the likelihood of) relapse of nicotine consumption following a period of nicotine abstinence (including abstinence in a subject who has achieved abstinence while being treated in accordance with the method), and/or may rescue a subject from relapse (including relapse in a subject who initially achieved abstinence while being treated in accordance with the method). Additionally, the methods described herein may permit the use of lower doses of nicotine receptor agonist and/or antagonist while still achieving efficacy as measured by the parameters discussed herein (for example, increased likelihood of quitting smoking, increased likelihood of achieving continuous abstinence and increased likelihood of achieving long-term abstinence), or may achieve efficacy in patients who may not otherwise be successful

The invention also includes embodiments where a subject's anti-nicotine antibody levels are monitored and maintained in order to promote long-term abstinence, as described in U.S. patent application Ser. No. 12/481,420 and PCT/US09/47679. In accordance with these embodiments, a subject's anti-nicotine antibody levels may be assessed after a successful quit attempt and/or after a successful period of abstinence, and a booster or subsequent course of nicotine immunogenic composition and/or anti-nicotine antibodies may be administered to maintain or return to the subject's antibody levels to a second minimum threshold level. The second minimum threshold level may be the same as or different from the first threshold level, and may be independently selected from the group consisting of at least about 6 μg/ml, at least about 10 μg/ml, at least about 12 μg/ml, at least about 15 μg/ml, at least about 20 μg/ml, at least about 25 μg/ml, at least about 30 μg/ml, at least about 35 μg/ml, at least about 40 μg/ml, at least about 45 μg/ml, and at least about 50 μg/ml, or from about 1.5 to about 2.0 times the subject's number of cigarettes smoked per day, such as at least 6 μg/ml, at least 10 μg/ml, at least 12 μg/ml, at least 15 μg/ml, at least 20 μg/ml, at least 25 μg/ml, at least 30 μg/ml, at least 35 μg/ml, at least 40 μg/ml, at least 45 μg/ml, and at least 50 μg/ml, or from 1.5 to 2.0 times the subject's number of cigarettes smoked per day.

In some embodiments, the methods further comprise administering anti-smoking counseling along with the combination drug treatment therapy described herein.

In alternative embodiments, as discussed in more detail above, the use of a nicotine immunogenic composition as described above is replaced by or supplemented with a passive immunization approach that involves the administration of anti-nicotine antibodies in addition to or in place of the nicotine immunogenic composition. In accordance with these embodiments, anti-nicotine antibodies are generated outside the body of the subject to be treated, in a suitable host mammal such as humans (in accordance with methods known in the art) and are administered to the subject.

In an exemplary embodiment, an immunogenic composition is administered to a subject followed by one or more boosters of the immunogenic composition. The subject's serum anti-nicotine antibody level is tested, and if the level is below a threshold level, a dose of an anti-nicotine antibody composition is administered. Following administration of the antibody composition, the subjects serum levels are again tested. If serum levels are still below threshold, another dose of the antibody composition is administered until serum levels reach the desired threshold. A target quit date can be selected to coincide with or follow a time by which the subject's serum anti-nicotine antibody level has reached a threshold level, as discussed above.

In specific embodiments, personalized medicine approaches are provided based on the subject's serum anti-nicotine antibody levels:

### Personalize Medicine Example 1

### Initial Therapy with Nicotine Immunogenic Composition

A subject may be administered a course of a nicotine immunogenic composition, and a first serum antibody testing date is selected to coincide with a time when the subject's serum anti-nicotine antibody levels are expected to reach a first threshold level, such as the exemplary first threshold serum antibody levels discussed above, e.g., at least about 10 μg/ml, at least about 12 μg/ml, at least about 15 μg/ml, at least about 20 μg/ml, at least about 25 μg/ml, at least about 30 μg/ml, at least about 35 μg/ml, at least about 40 μg/ml, at least about 45 μg/ml, or, at least about 50 μg/ml, or threshold serum anti-nicotine antibody levels that are (in μg/ml) from at least about 1.5 to at least about 2.0 times the number of cigarettes smoked per day by the subject, such as being 1.5 to 2.0 times the number of cigarettes smoked per day. In other embodiments, the first minimum threshold level is directly correlated with the number of doses of a nicotine immunogenic composition that the subject has received prior to the measuring of the level of anti-nicotine antibodies. For instance, the first specified threshold anti-nicotine antibody level can be selected from at least about 10 μg/ml for up to two prior doses (such as from at least 10 to at least 25 μg/ml), at least about 25 μg/ml for three prior doses (such as from at least 25 to at least 50 μg/ml), at least about 50 μg/ml for four prior doses (such as from at least 50 to at least 75 μg/ml), and at least about 60 μg/ml for five or more prior doses (such as from at least 60 to at least 75 or at least 100 μg/ml). The practitioner readily can determine corresponding secreted antibody levels using routine methodologies.

The first serum antibody testing date may be a few weeks after the first dose of nicotine immunogenic composition is administered, such as two or more weeks after the first dose of nicotine immunogenic composition is administered, depending on the immunogenic potency of nicotine immunogenic composition and the first threshold serum antibody. For example, in embodiments where the course of nicotine immunogenic composition comprises an initial dose followed by four or five booster doses, the first serum antibody testing date may fall about one week after the first booster dose (second absolute dose) is administered.

If the subject's serum anti-nicotine antibody levels are below the first threshold level at the first serum antibody testing date, the method may further comprise one or more of the following: (i) administering another dose of a nicotine immunogenic composition; (ii) administering anti-nicotine antibodies; or (iii) administering a course of a nicotine receptor agonist and/or antagonist.

If another dose of a nicotine immunogenic composition is administered, it may be administered in accordance with the original dosing schedule or according to a revised dosing schedule, that may result in the next dose being provided sooner or later than in accordance with the original dosing schedule. For example, delaying the next dose of nicotine immunogenic compositions may result in an enhanced immune response and/or a greater level of anti-nicotine antibodies achieved in the subject. In specific embodiments, the next dose is delayed for a period of about one week, about two weeks, about three weeks, about four weeks, about five weeks, about six weeks, or longer, such as being delayed for a period of one week, two weeks, three weeks, four weeks, five weeks, six weeks, or longer. Additionally or alternatively, the nicotine immunogenic composition may be the same or different from an already administered nicotine immunogenic composition, such as by comprising one or more of a different dose, antigen, or adjuvant, or by being formulated in a different carrier composition or for a different route of administration.

If another dose of a nicotine immunogenic composition is administered, or if an anti-nicotine antibody composition is administered, a second serum antibody testing date may be selected to further aid selection of a target quit date. Alternatively, if an anti-nicotine antibody composition is administered, the dose may be selected to achieve the first threshold level. In that case, the date of administration of the antibody composition may become a target quite date and/or the subject may be counseled to quit smoking (or cease nicotine consumption) on or after that date. If a course of a nicotine receptor agonist and/or antagonist is administered, a target quit date may be selected in accordance with the specific nicotine receptor agonist and/or antagonist, such as being one week after commencing a course of nicotine agonist and/or antagonist (such as varenicline), or some other time during the course of nicotine agonist and/or antagonist (such as varenecline), with or without further testing of the subject's serum anti-nicotine antibody level.

If the subject's serum anti-nicotine antibody levels are at or above the first threshold level at the first serum antibody testing date, that date may become a target quite date and/or the subject may be counseled to quit smoking (or cease nicotine consumption) on or after that date.

If a second serum antibody testing date is selected, and the subject's serum anti-nicotine antibody levels are still below the target threshold level, the method again may further comprise one or more of (i) administering another dose of a nicotine immunogenic composition; (ii) administering anti-nicotine antibodies; or (iii) administering a course of a nicotine receptor agonist and/or antagonist, as described above. As noted above, the threshold level of serum anti-nicotine antibodies may be selected based on the number of doses of a nicotine immunogenic composition that the subject has received prior to the measuring of the level of anti-nicotine antibodies. Thus, the target threshold level for a second (or subsequent) serum antibody testing date may be different from that for a first (or previous) serum antibody testing date, particularly if the subject was administered a further dose of a nicotine immunogenic composition on or after the first (or previous) serum antibody testing date.

At any time, the further therapy may be the same or different as administered previously, such as administering the same or different nicotine immunogenic composition, the same or different antibody composition, and/or the same or different nicotine receptor agonist and/or antagonist, or the same or different combination of one or more of these, in any permutation or combination thereof. The selection of serum antibody testing dates and selection of further therapies may be repeated until a target threshold level of serum anti-nicotine antibodies is reached, or until the subject quits smoking (or ceases nicotine consumption).

If the subject's serum anti-nicotine antibody levels are at or above the target threshold level at the second (or subsequent) serum antibody testing date, that date may become a target quite date and/or the subject may be counseled to quit smoking (or cease nicotine consumption) on or after that date.

In accordance with any of these embodiments, an anti-nicotine antibody composition can be used in place of one or more doses of nicotine immunogenic composition, as described above.

### Personalize Medicine Example 2

### Initial Therapy with Nicotine Immunogenic Composition & Anti-Nicotine Agent

As another example, a subject may be administered a course of a nicotine immunogenic composition and a course of a nicotine agonist and/or antagonist as described herein, and a first serum antibody testing date is selected to coincide with a time during the course of treatment with the nicotine agonist and/or antagonist, when the subject's serum anti-nicotine antibody levels are expected to reach a first threshold level, such as the exemplary first threshold serum antibody levels discussed above. The first serum antibody testing date may be about one week after commencing the course of nicotine agonist and/or antagonist, or some other time during the course of nicotine agonist and/or antagonist, as may be appropriate depending on the specific nicotine agonist and/or antagonist.

If the subject's serum anti-nicotine antibody levels are below the first threshold level at the first serum antibody testing date, the method may further comprise one or more of the following, as described above: (i) administering another dose of a nicotine immunogenic composition; (ii) administering anti-nicotine antibodies; or (iii) administering a course of a nicotine receptor agonist and/or antagonist. As in the embodiments described above, if another dose of a nicotine immunogenic composition is administered, it may be administered in accordance with the original dosing schedule or according to a revised dosing schedule, that may result in the next dose being provided sooner or later than in accordance with the original dosing schedule. Moreover, the nicotine immunogenic composition may be the same or different from the already administered nicotine immunogenic composition, as described above.

As described above, if another dose of a nicotine immunogenic composition is administered, or if an anti-nicotine antibody composition is administered, a second serum antibody testing date may be selected to further aid selection of a target quit date. Alternatively, if an anti-nicotine antibody composition is administered, the dose may be selected to achieve the first threshold level. In that case, the date of administration of the antibody composition may become a target quite date and/or the subject may be counseled to quit smoking (or cease nicotine consumption) on or after that date. If a course of a nicotine receptor agonist and/or antagonist is administered, the nicotine receptor agonist and/or antagonist may be the same or different as the one(s) previously administered. Moreover, if a course of a nicotine receptor agonist and/or antagonist is administered, a target quit date may be selected in accordance with the specific nicotine receptor agonist and/or antagonist, such as being one week after commencing the course of nicotine agonist and/or antagonist, or some other time during the course of nicotine agonist and/or antagonist, with or without further testing of the subject's serum anti-nicotine antibody level.

If the subject's serum anti-nicotine antibody levels are at or above the first threshold level at the first serum antibody testing date, that date may become a target quite date and/or the subject may be counseled to quit smoking (or cease nicotine consumption) on or after that date.

As in the embodiments described above, if a second serum antibody testing date is selected, and the subject's serum anti-nicotine antibody levels are still below the target threshold level, the method again may further comprise one or more of the same options outlined above, in any permutation or combination thereof. The selection of serum antibody testing dates and selection of further therapies may be repeated until a first threshold level of serum anti-nicotine antibodies is reached, or until the subject quits smoking (or ceases nicotine consumption). As noted above, the threshold of serum anti-nicotine antibodies may be selected based on the number of doses of a nicotine immunogenic composition that the subject has received prior to the measuring of the level of anti-nicotine antibodies. Thus, the target threshold level for a second or subsequent serum antibody testing date may be different from that for a first or previous serum antibody testing date, particularly if the subject was administered a further dose of a nicotine immunogenic composition on or after the first or previous serum antibody testing date. If the subject's serum anti-nicotine antibody levels are at or above the target threshold level at the second (or subsequent) serum antibody testing date, that date may become a target quite date and/or the subject may be counseled to quit smoking (or cease nicotine consumption) on or after that date.

In accordance with any of these embodiments, an anti-nicotine antibody composition can be used in place of one or more doses of nicotine immunogenic composition, as described above.

### V. KITS

Also disclosed herein are kits useful for treating nicotine addiction and nicotine-addiction related disorders, promoting smoking cessation, extending the duration of smoking abstinence in a subject who has quit smoking, increasing the likelihood of long-term abstinence, preventing relapse (e.g., reducing the likelihood of a relapse) of nicotine consumption following a period of nicotine abstinence, and/or rescuing a subject from relapse.

In some embodiments, the kits include at least one dose of a nicotine immunogenic composition and/or a anti-nicotine antibody composition, at least one dose of a nicotine receptor agonist or antagonist, and instructions for administering the dose(s) of the nicotine immunogenic composition and/or anti-nicotine antibody composition and the dose(s) of the nicotine receptor agonist and/or a nicotine receptor antagonist in overlapping courses.

In other embodiments, the kits include at least one dose of a nicotine immunogenic composition and/or a anti-nicotine antibody composition and instructions for administering the dose(s) of the nicotine immunogenic composition and/or an anti-nicotine antibody composition in conjunction with a course of a nicotine receptor agonist and/or a nicotine receptor antagonist.

In other embodiments, the kits include at least one dose of a nicotine receptor agonist and/or a nicotine receptor antagonist and instructions for administering the dose(s) of the nicotine receptor agonist and/or the nicotine receptor antagonist in conjunction with a course of a nicotine immunogenic composition and/or a anti-nicotine antibody composition.

In other embodiments, the kits include one or more of (i) one or more dose(s) for a course of a nicotine immunogenic composition and/or a anti-nicotine antibody composition and (ii) one or more dose(s) for a course of a nicotine receptor agonist and/or a nicotine receptor antagonist, along with instructions for administering the nicotine immunogenic composition and/or a anti-nicotine antibody composition and the nicotine receptor agonist and/or the nicotine receptor antagonist in overlapping courses.

In other embodiments, the kits include (a) at least one dose of one or more of (i) a nicotine immunogenic composition and (ii) anti-nicotine antibody composition; (b) at least one dose of a nicotine receptor agonist and/or nicotine receptor antagonist; and (c) instructions for administering to the subject the dose(s) of the nicotine immunogenic composition and/or anti-nicotine antibody composition and the dose(s) of the nicotine receptor agonist and/or nicotine receptor antagonist in overlapping courses.

In other embodiments, the kits include at least one dose of a nicotine immunogenic composition and at least one does of an anti-nicotine antibody composition and instructions for administering the dose(s) of the nicotine immunogenic composition and/or an anti-nicotine antibody composition. For example, the instructions may indicate administering to the subject the dose(s) of the nicotine immunogenic composition and anti-nicotine antibody composition in order to achieve a threshold serum anti-nicotine antibody level, wherein threshold serum anti-nicotine antibody level may be any threshold serum anti-nicotine antibody level described above.

In any of these embodiments, the instructions may indicate that the relative timing of the courses of nicotine immunogenic composition and/or a anti-nicotine antibody composition and nicotine receptor agonist and/or a nicotine receptor antagonist is as described above, e.g., is such that a first threshold level of anti-nicotine antibodies is achieved in the subject by the time that the course of the nicotine receptor agonist or antagonist is completed and/or by the time that the efficacy of the nicotine receptor agonist or antagonist is diminishing or has diminished, as discussed in more detail above.

In any of these embodiments, the kit may further include instructions for selecting a target quit date, including any one or more target quit dates selected as described above. In some embodiments, the instructions indicate that the target quit date should be about 1-2 weeks after the first dose of nicotine receptor agonist or antagonist, as discussed above. In some embodiments, the target quit date is about 1 week, 2 weeks, or longer, after the first dose of nicotine immunogenic composition and/or a anti-nicotine antibody composition, or before, after, or coincident with any subsequent dose of nicotine immunogenic composition and/or a anti-nicotine antibody composition, as discussed above. In some embodiments, the target quit date is determined with reference to the subject's level of anti-nicotine antibodies, as discussed above. In accordance with the latter embodiments, or with any other embodiments described herein, the kit may further comprise components useful for assessing the subject's anti-nicotine antibody levels and further instructions for use, as described in U.S. patent application Ser. No. 12/481,420 and international patent application PCT/US09/47679.

The embodiments of the methods and kits described herein are not intended to be limiting. Thus, for example, any of the embodiments specifically described can be combined with one or more other embodiments also specifically described. All of these combinations and permutations are contemplated as part of the invention.

The following specific examples are included as illustrative only. These examples are in no way intended to limit the scope of the invention. Other aspects of the invention will be apparent to those skilled in the art to which the invention pertains.

### VI. EXAMPLES

### A. NicVAX® & Varenicline

A double-blind, placebo-controlled, co-administration study (add-on study) will be performed with a combination of NicVAX® and varenicline or varenicline plus a vaccine-placebo. The study will be conducted with 600 human subjects (both men and women) between the ages 18-65. All subjects will be heavy smokers, with no subject smoking less than 10 cigarettes per day. Also, subjects will not have succeeded in quitting smoking for three months in the past year. The design of the study is shown in FIG. 2.

As shown in FIG. 2, the subjects will be randomly divided into one of two arms. Subjects in one arm of the study will receive a combination of varenicline plus NicVAX®. Subjects in the other arm will receive varenicline plus a vaccine-placebo (phosphate buffered saline and alum).

Vaccinations (either NicVAX® or placebo) will be given at time −2, then at weeks 2, 6, 10, 14 and 24, relative to the first administration of varenicline. 400 μg of NicVAX will be administered at each scheduled vaccination. Two weeks after the first vaccination, both groups will begin a 12 week course of daily doses of varenicline (CHANTIX®/CHAMPIX). CHANTIX® will be administered according to the manufacturer's instructions (i.e., Days 1-3: 0.5 mg once daily; Days 4-7: 0.5 mg twice daily; Days 8-End of treatment at 12 weeks: 1 mg twice daily).

The target quit date (TQD) is planned for three weeks after the first vaccination, e.g., one week after the first dose of varenicline. The target quit date and the counseling regimen will be identical for both aims of the study, eliminating the biases that are problematic in double-dummy comparator designs.

The primary clinical endpoint of the study will be continuous abstinence from smoking during weeks 19-26 after first vaccination. Secondary endpoints will include abstinence in weeks 9-12 and/or abstinence in weeks 37-52.

Results

The study is underway, but because it is a blinded study results are not yet available. The study will show that the combination of NicVAX® and varenicline provides improved efficacy as compared with varenicline or NicVAX® alone, and that vaccination with NicVAX® reduces the relapse rate for subjects initially successfully treated with varenicline.

### B. NicVAX® & Bupropion

A similar study can be designed and performed with a combination of NicVAX® and bupropion. The study will show that the combination of NicVAX® and bupropion provides improved efficacy as compared with bupropion or NicVAX® alone, and that vaccination with NicVAX® reduces the relapse rate for subjects initially successfully treated with bupropion.

1. A method for treating nicotine addiction in a subject comprising:
(a) inducing a threshold level of anti-nicotine antibodies in a subject by administering one or more of (i) a nicotine immunogenic composition and (ii) a composition comprising anti-nicotine antibodies; and
(b) administering a course of a nicotine receptor agonist or a nicotine receptor antagonist to the subject;
wherein the threshold level of anti-nicotine antibodies is achieved in the subject by the time that the course of nicotine receptor agonist or nicotine receptor antagonist is completed.

2. The method of claim 1, wherein step (a) comprises administering a course of a nicotine immunogenic composition to the subject, and step (b) comprises administering a course of a nicotine receptor agonist to the subject.

3. The method of claim 1, wherein the threshold level of anti-nicotine antibodies is selected from the group consisting of at least about 6 μg/ml, at least about 10 μg/ml, at least about 12 μg/ml, at least about 15 μg/ml, at least about 20 μg/ml, at least about 25 μg/ml, at least about 30 μg/ml, at least about 35 μg/ml, at least about 40 μg/ml, at least about 45 μg/ml, and at least about 50 μg/ml.

4. The method of claim 1, wherein step (a) comprises administering a course of a nicotine immunogenic composition to the subject, and the threshold level of anti-nicotine antibodies is directly correlated with the number of doses of a nicotine immunogenic composition that the subject has received and is selected from the group consisting of at least 10 μg/ml for a subject who has received up to two doses of a nicotine immunogenic composition; at least 25 μg/ml for a subject who has received three doses of a nicotine immunogenic composition; at least 50 μg/ml for a subject who has received four doses of a nicotine immunogenic composition, and at least 60 μg/ml for a subject who has received five or more doses of a nicotine immunogenic composition.

5. The method of claim 2, wherein the threshold level of anti-nicotine antibodies is attained by about the 6th week through about the 12th week of the course the nicotine receptor agonist.

6. The method of claim 2, wherein the nicotine immunogenic composition is administered according to a course that comprises the administration of one to six doses of the nicotine immunogenic composition over about a six month period.

7. The method of claim 2, wherein the course (b) of the nicotine receptor agonist comprises the administration of at least a daily dose of the nicotine receptor agonist over a 12-week period.

8. The method of claim 2, wherein the nicotine immunogenic composition is administered before the course (b) of the nicotine receptor agonist is started.

9. The method of claim 1, wherein the method comprises administering a composition comprising anti-nicotine antibodies substantially simultaneously with the course of nicotine receptor agonist or nicotine receptor antagonist.

10. The method of claim 1, wherein the method comprises administering a composition comprising anti-nicotine antibodies near the end of the course of nicotine receptor agonist or nicotine receptor antagonist.

11. The method of claim 8, wherein a first dose of the course of the nicotine immunogenic composition is administered to the subject at least 2 weeks before a first dose of the course of the nicotine receptor agonist.

12. The method of claim 1, further comprising selecting a target quit date that is about one week after the first dose of nicotine receptor agonist or nicotine receptor antagonist is administered.

13. The method of claim 1, further comprising selecting a target quit date when the subject's anti-nicotine antibody levels are at least at the threshold level.

14. The method of claim 1, wherein the nicotine immunogenic composition comprises a nicotine-carrier conjugate comprising a nicotine hapten conjugated to a suitable carrier protein.

15. The method of claim 1, wherein the nicotine immunogenic composition comprises a nicotine-carrier conjugate comprising 3′aminomethylnicotine conjugated to a suitable carrier protein.

16. The method of claim 2, wherein the nicotine immunogenic composition comprises a nicotine-carrier conjugate comprising 3′aminomethylnicotine conjugated to a suitable carrier protein.

17. The method of claim 1, wherein the nicotine receptor agonist or nicotine receptor antagonist comprises varenicline.

18. The method of claim 2, wherein the nicotine receptor agonist comprises varenicline.

19. The method of claim 1, wherein step (a) comprises administering a course of a nicotine immunogenic composition to the subject, and step (b) comprises administering a course of a nicotine receptor antagonist to the subject.

20. The method of claim 19, wherein the threshold level of anti-nicotine antibodies is achieved in the subject by the time that the efficacy of the nicotine receptor antagonist is diminishing.

21. The method of claim 19, wherein the nicotine receptor antagonist comprises bupropion.

22. The method of claim 1, wherein the nicotine receptor agonist or nicotine receptor antagonist comprises bupropion.

23. A method for extending the duration of smoking abstinence in a subject who has quit smoking, increasing the likelihood of long-term abstinence from smoking, promoting smoking cessation in a subject, or preventing relapse of nicotine consumption following a period of nicotine abstinence in a subject, comprising:
(a) inducing a threshold level of anti-nicotine antibodies in a subject by administering one or more of (i) a nicotine immunogenic composition and (ii) a composition comprising anti-nicotine antibodies; and
(b) administering a course of a nicotine receptor agonist or a nicotine receptor antagonist to the subject;
wherein the threshold level of anti-nicotine antibodies is achieved in the subject by the time that the course of nicotine receptor agonist or nicotine receptor antagonist is completed.

24. A kit for treating nicotine addiction in a subject comprising:
(a) at least one dose of one or more of a nicotine immunogenic composition and an anti-nicotine antibody composition;
(b) at least one dose of a nicotine receptor agonist and/or nicotine receptor antagonist; and
(c) instructions for administering to the subject the dose(s) of the nicotine immunogenic composition and/or anti-nicotine antibody composition and the dose(s) of the nicotine receptor agonist and/or nicotine receptor antagonist in overlapping courses such that a threshold level of anti-nicotine antibodies is achieved in the subject by the time that the course of nicotine receptor agonist or nicotine receptor antagonist is completed.

25. The kit of claim 24, comprising at least one dose of a nicotine immunogenic composition.

26. The kit of claim 25, further comprising at least one dose of an anti-nicotine antibody composition.

27. A method for treating nicotine addiction in a subject comprising:
(a) administering a nicotine immunogenic composition to the subject
(b) measuring the subject's serum anti-nicotine antibody level; and
(c) if the measured serum anti-nicotine antibody level is below a threshold level, administering anti-nicotine antibodies.

28. The method of claim 27, wherein the threshold level of anti-nicotine antibodies is selected from the group consisting of at least about 10 μg/ml, at least about 12 μg/ml, at least about 15 μg/ml, at least about 20 μg/ml, at least about 25 μg/ml, at least about 30 μg/ml, at least about 35 μg/ml, at least about 40 μg/ml, at least about 45 μg/ml, and at least about 50 μg/ml.

29. The method of claim 27, wherein the threshold level of anti-nicotine antibodies is directly correlated with the number of doses of a nicotine immunogenic composition that the subject has received and is selected from the group consisting of at least 10 μg/ml for a subject who has received up to two doses of a nicotine immunogenic composition; at least 25 μg/ml for a subject who has received three doses of a nicotine immunogenic composition; at least 50 μg/ml for a subject who has received four doses of a nicotine immunogenic composition, and at least 60 μg/ml for a subject who has received five or more doses of a nicotine immunogenic composition.

30. The method of claim 28, further comprising administering a nicotine receptor agonist and/or a nicotine receptor antagonist.

31. A kit for treating nicotine addiction in a subject comprising:
(a) at least one dose of a nicotine immunogenic composition;
(b) at least one dose of an anti-nicotine antibody composition; and
(c) instructions for administering to the subject the dose(s) of the nicotine immunogenic composition and anti-nicotine antibody composition to achieve a threshold serum anti-nicotine antibody level.

32. A method for treating nicotine addiction in a subject comprising:
(a) administering one or more of a first nicotine immunogenic composition and a first anti-nicotine antibody composition;
(b) measuring the level of anti-nicotine antibodies in serum from said subject;
(c) if the measured serum anti-nicotine antibody level is below a threshold level, administering one or more of:
(i) a second nicotine immunogenic composition; (ii) a second anti-nicotine antibody composition; and (iii) a nicotine receptor agonist and/or antagonist.

33. The method of claim 32, wherein step (a) further comprises administering a nicotine receptor agonist and/or nicotine receptor antagonist.

34. The method of claim 32, further comprising, after step (c):
(d) measuring the level of anti-nicotine antibodies in serum from said subject;
(e) if the measured serum anti-nicotine antibody level is below a threshold level, administering one or more of:
(i) a nicotine immunogenic composition; (ii) anti-nicotine antibodies; and (iii) a nicotine receptor agonist and/or antagonist.
